(19) United States
(12) Patent Application Publication
(10) Pub. No.: US 2020/0022997 A1
(43) Pub. Date: Jan. 23, 2020

(54) ORAL ANTICANCER PRODRUGS, THEIR PREPARATION METHODS AND APPLICATIONS

(71) Applicants: Seoul National university R&DB Foundation, Seoul (KR); University of Ulsan Foundation For Industry Cooperation, Ulsan (KR); The Asia Foundation, Seoul (KR)

(72) Inventors: Youngro BYUN, Seoul (KR); Sang Yoon KIM, Seoul (KR); Seongwoo CHUNG, Seoul (KR); Young Seok CHO, Seoul (KR)

(73) Assignees: Seoul National university R&DB Foundation, Seoul (KR); University of Ulsan Foundation For Industry Cooperation, Ulsan (KR); The Asia Foundation, Seoul (KR)

(21) Appl. No.: 16/435,680

(22) Filed: Jun. 10, 2019

(30) Foreign Application Priority Data
Jun. 11, 2018 (KR) 10-2018-0066908

(51) Int. Cl. A61K 31/704; A61K 31/337; A61K 47/54; A61K 47/55; A61K 47/64; A61P 35/00

(52) U.S. Cl. CPC A61K 31/704; A61K 31/337; A61K 47/554; A61K 45/06; A61K 47/64; A61P 35/00; A61K 47/55

(57) ABSTRACT

Described herein are anticancer complexes suitable for oral administration comprising an anticancer prodrug moiety comprising (i) a caspase-cleavable peptide, attached directly or through a linker, to (ii) an anticancer chemotherapeutic agent; and (b) a bile acid moiety, wherein the bile acid moiety is non-covalently complexed to the anticancer prodrug moiety. Also described is the preparation of such anticancer complexes and their use in combination with a treatment that induces apoptosis (e.g., radiotherapy) for inducing amplified apoptosis of tumor cells and treating cancer.

### CROSS-REFERENCE TO RELATED APPLICATIONS

This application claims priority to Korean application 10-2018-0066908 filed Jun. 11, 2018, the entire contents of which are incorporated herein by reference.

### FIELD OF INVENTION

Described herein are anticancer complexes comprising an anticancer prodrug moiety and a bile acid moiety that can be orally administered. Also described are the preparation of such anticancer complexes and their use in combination with a treatment that induces apoptosis (e.g., radiotherapy or chemotherapy) for treating cancer.

### BACKGROUND

Anti-cancer chemotherapeutic agents (or anticancer drugs) are widely used in cancer treatment for their powerful anticancer therapeutic effects; nevertheless, their use is often limited by serious side effects and toxicity. Efforts to develop selective chemotherapy that targets tumors as opposed to healthy tissue have focused on targeted delivery of chemotherapeutic agents to tumor cells, such as by using antibodies or peptides that recognize and bind to molecules expressed preferentially in tumor cells but not normal cells. However, wide use of such drugs is limited since such drugs can only be administered to patients whose tumor cells express the target molecules. Moreover, recent research has shown that even within a given patient tumor cells may exhibit intratumoral heterogeneity, with phenotypes differing among cancer cells within a single tumor tissue. This means that even if a biopsy confirms the expression of the target molecule for a given drug, all tumor cells may not express the target, thereby limiting therapeutic effectiveness.

Thus, there remains a need for chemotherapeutic agents that are selective for tumor cells and do not harm normal tissue but are effective against broad range of tumor cells with different phenotype at the same time.

Furthermore, while many chemotherapeutic agents are administered intravenously, intravenous administration has its limitations. Patent Registration of the Republic of Korea No. 10-1759261 describes an anticancer prodrug conjugate that binds to albumin and was formulated for parenteral injection. Binding to albumin increased the half-life of the chemotherapeutic agent in the blood and reduced the frequency of treatment. However, intravenous administration is accompanied with an increased risk of infection and low patient convenience and compliance. Accordingly, it is difficult in practice to administer chemotherapeutic agents on an ongoing basis as may be required to maintain therapeutic levels for a persistent therapeutic effect.

Therefore, there is a need for oral anticancer chemotherapeutic agents that can be absorbed in the gastrointestinal tract and exhibit low toxicity. Oral administration is an attractive approach since patients can continuously and repeatedly administer the drug by him/herself and thereby maintain therapeutic levels for a persistent therapeutic effect.

### SUMMARY

Provided herein are complexes comprising:
(a) an anticancer prodrug moiety comprising (i) a caspase-cleavable peptide, covalently attached directly or through a linker, to (ii) an anticancer chemotherapeutic agent; and
(b) a bile acid moiety, wherein the bile acid moiety is non-covalently complexed to the anticancer prodrug moiety.

In some embodiments, the caspase-cleavable peptide is cleavable by a caspase selected from caspase-3, caspase-7, and caspase-9. In some embodiments, the caspase-cleavable peptide comprises an amino acid sequence selected from Asp-Glu-Val-Asp (SEQ ID NO:4), Asp-Leu-Val-Asp (SEQ ID NO:5), Asp-Glu-Ile-Asp (SEQ ID NO:6), and Leu-Glu-His-Asp (SEQ ID NO:7). In some embodiments, the caspase-cleavable peptide comprises the amino acid sequence Asp-Glu-Val-Asp (SEQ ID NO:4).

In some embodiments, the caspase-cleavable peptide is covalently attached to the anticancer chemotherapeutic agent through a linker selected from para-aminobenzyloxycarbonyl, aminoethyl-N-methylcarbonyl, aminobiphenylmethyloxycarbonyl, a dendritic linker and a cephalosporin-based linker. In some embodiments, the caspase-cleavable peptide is covalently attached to the anticancer chemotherapeutic agent through a para-aminobenzyloxycarbonyl linker.

In some embodiments, the anticancer chemotherapeutic agent is selected from anthracyclines, antibiotics, alkylating agents, platinum-based agents, antimetabolites, topoisomerase inhibitors, and mitotic inhibitors. In some embodiments, the anticancer chemotherapeutic agent is selected from doxorubicin, daunorubicin, epirubicin, idarubicin, valrubicin, anthracyclin; actinomycin-D, bleomycin, mitomycin-C, cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine; camptothecin, topotecan, irinotecan, etoposide, teniposide, mitoxantrone, paclitaxel, docetaxel, izabepilone, vinblastine, vincristine, vindesine, vinorelbine, estramustine, and derivatives thereof. In some embodiments, the anticancer chemotherapeutic agent is selected from doxorubicin, daunorubicin, and docetaxel.

In some embodiments, the bile acid moiety is selected from cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, and hyodeoxycholic acid.

In some embodiments, the bile acid moiety is N^{α}-deoxycholyl-L-lysine-methylester. In some embodiments, a plurality of bile acid moieties is non-covalently complexed to a single anticancer prodrug moiety. In some embodiments, the bile acid moiety is non-covalently complexed to the anticancer prodrug moiety by ionic bonding, hydrophobic bonding, or coordinate bonding.

In some embodiments, the (a) the anticancer prodrug moiety comprises (i) a caspase-cleavable peptide comprising the amino acid sequence Asp-Glu-Val-Asp joined through a para-aminobenzyloxycarbonyl linker to (ii) an anticancer chemotherapeutic agent; and (b) the bile acid moiety comprises N^{α}-deoxycholyl-L-lysine-methylester.

Also provided herein are oral pharmaceutical compositions comprising any one of the complexes described herein and a pharmaceutically acceptable carrier for oral administration.

Also provided herein is a method of inducing amplified apoptosis of tumor cells in a subject, comprising:
(a) administering to a subject in need thereof an apoptosis-inducing treatment effective to induce expression of caspase in tumor cells, and
(b) orally administering to the subject any one of the complexes described herein.

In some embodiments, the apoptosis-inducing treatment is selected from radiotherapy, hyperthermia, laser therapy, photodynamic therapy, chemotherapy, and cryosurgery. In some embodiments, the method comprises weekly administration of radiotherapy, such as at a dose of 1 to 35 Gy, and daily administration of the complex, such as at a dose of 1 to 100 mg/kg, based on the molar equivalent dose of the anticancer chemotherapeutic agent. In some embodiments, the method comprises weekly administration of radiotherapy at a dose of 2 to 10 Gy, and daily administration of the complex at a dose of 1 to 10 mg/kg, based on the molar equivalent dose of the anticancer chemotherapeutic agent. In some embodiments, the method results in gastrointestinal absorption of the chemotherapeutic prodrug.

In some embodiments, the apoptosis-inducing treatment comprises chemotherapy, such as chemotherapy with olaparib, trastzumab, ado-trastuzumab emtansine, tamoxifen, lapatinib, palbociclib, ribociclib, neratinib maleate, or abemaciclib. In some embodiments, the apoptosis-inducing treatment comprises chemotherapy with olaparib.

The foregoing summary is illustrative only and is not intended to be in any way limiting. In addition to the illustrative aspects, embodiments, and features described above, further aspects, embodiments, and features will become apparent by reference to the detailed description and examples herein below.

### BRIEF DESCRIPTION OF THE DRAWINGS

FIG. 1 depicts a mode of action for the DEVD-S-DOX/DCK complex in a radiotherapy-assisted orally available metronomic apoptosis-targeted chemotherapy.

FIG. 2 depicts a synthesis for preparing the DEVD-S-DOX/DCK complex.

FIG. 3 depicts the chemical structure of the DEVD-S-DOX/DCK complex.

FIG. 4 depicts the DSC (differential scanning calorimetry) thermograms of DEVD-S-DOX, and DCK, DEVD-S-DOX/DCK.

FIG. 5 depicts the in vitro concentration dependent cytotoxicity of doxorubicin, DEVD-S-DOX and DEVD-S-DOX preincubated with caspase-3 for MDA-MB-231 breast cancer cells in vitro.

FIG. 6A depicts the determination of caspase-3 upregulation after radiation exposure (4 Gy) in MDA-MB-231 breast cancer cells by using Western blot.

FIG. 6B depicts the determination of caspase-3 upregulation after radiation exposure (4 Gy) in MDA-MB-231 breast cancer cells by using cellular caspase-3 activity assay. Data are presented as mean±SEM. **p<0.01 and ***p<0.001 versus control or as indicated.

FIG. 7 depicts the cytotoxicity of DEVD-S-DOX and doxorubicin with and without radiation (4 Gy) and/or caspase inhibitor (Z-VAD-FMK) in MDA-MB 231 breast cancer cells in vitro. Data are presented as mean±SEM. **p<0.01 and ***p<0.001 versus control or as indicated.

FIG. 8 depicts cellular uptake of DEVD-S-DOX and doxorubicin with and without radiation (4 Gy) and/or caspase inhibitor (Z-VAD-FMK) in MDA-MB 231 breast cancer cells in vitro by fluorescence microscopy.

FIG. 9 depicts the plasma drug concentration profiles of DEVD-S-DOX and DEVD-S-DOX/DCK complex in SD rats after intravenous and oral administration. Doses are doxorubicin molar equivalent dose and data are presented as ±SEM.

FIG. 10 depicts the confocal microscopy images of sectioned GI tract isolated from a SD rat that received DEVD-S-DOX/DCK complex orally. The scale bar is 100 μm.

FIG. 11 depicts the cellular absorption of DEVD-S-DOX and DEVD-S-DOX/DCK complex in Caco-2 cells (red). Tight junction and nucleus were stained with phalloidin FITC (green) and DAPI (blue), respectively. The scale bar is 50 μm.

FIG. 12 depicts the cellular uptake of DEVD-S-DOX and DEVD-S-DOX/DCK complex on MDCK and hASBT-MDCK cells. The scale bar is 50 μm.

FIG. 13 depicts the tumor growth profiles of MDA-MB-231 xenografted mice that received saline as a control, single dose radiation, DEVD-S-DOX with radiation, and DEVD-S-DOX/DCK complex with and without radiation in a pre-clinical model of breast cancer. The drugs were orally administered daily. The radiation was given at a dose of 4 Gy, and the arrows depict the day when radiation was given. Data are presented as mean±SEM. *p<0.05, **p<0.01, and ***p<0.001 versus control or as indicated.

FIG. 14A depicts the tumor growth of MDA-MB-231 xenografted mice that received saline as a control, repeated-dose radiotherapy, and DEVD-S-DOX/DCK complex (5 mg/kg) coupled with repeated-dose radiation. The drugs were orally administered daily. The radiation was given at a dose of 4 Gy, and the arrows depict the day when radiation was given. Data are presented as mean±SEM. *p<0.05, **p<0.01, and ***p<0.001 versus control or as indicated.

FIG. 14B depicts the body weight profiles of MDA-MB-231 xenografted mice that received saline as a control, repeated-dose radiotherapy, and DEVD-S-DOX/DCK complex (5 mg/kg) coupled with repeated-dose radiation.

FIG. 15A depicts the tumor growth of HCC-70 xenografted mice that received saline as a control, repeated-dose radiotherapy, and DEVD-S-DOX/DCK complex (5 mg/kg) coupled with repeated-dose radiation over a period of 20 days. The drugs were orally administered daily. The radiation was given at a dose of 4 Gy, and the arrows depict the day when radiation was given. Data are presented as mean±SEM. *p<0.05, **p<0.01, and ***p<0.001 versus control or as indicated.

FIG. 15B depicts the body weight profiles of HCC-70 xenografted mice that received saline as a control, repeated-dose radiotherapy, and DEVD-S-DOX/DCK complex (5 mg/kg) coupled with repeated-dose radiation over a period of 20 days.

FIG. 16A shows the tumor volume profile of Pan02 grafted mice that received saline as a control, repeated-dose radiotherapy, and DEVD-S-DOX/DCK complex (10 mg/kg) coupled with repeated-dose radiation. The drugs were orally administered daily. The radiation was given at a dose of 4 Gy at day 0 and day 7. Data are presented as mean±SEM. **p<0.01, and ***p<0.001 versus control or as indicated.

FIG. 16B shows the body weight profile of Pan02 grafted mice that received saline as a control, repeated-dose radiotherapy, and DEVD-S-DOX/DCK complex (10 mg/kg) coupled with repeated-dose radiation.

FIG. 16C shows the tumor weight of Pan02 grafted mice that received saline as a control, repeated-dose radiotherapy, and DEVD-S-DOX/DCK complex (10 mg/kg) coupled with repeated-dose radiation.

FIG. 16D shows the tumors of Pan02 grafted mice that received saline as a control, repeated-dose radiotherapy, and DEVD-S-DOX/DCK complex (10 mg/kg) coupled with repeated-dose radiation.

FIG. 17A shows the tumor volume profile of HCC-70 xenografted mice that received saline as a control, repeated-dose radiotherapy, and DEVD-S-DOX/DCK complex (5 mg/kg) coupled with repeated-dose radiation over a period of 30 days. The drugs were orally administered daily. The radiation was given at a dose of 4 Gy at day 0 and day 7. Data are presented as mean±SEM. **p<0.01, and ***p<0.001 versus control or as indicated.

FIG. 17B shows the body weight profile of HCC-70 xenografted mice that received saline as a control, repeated-dose radiotherapy, and DEVD-S-DOX/DCK complex (5 mg/kg) coupled with repeated-dose radiation over a period of 30 days.

FIG. 17C shows the tumor weight of HCC-70 xenografted mice that received saline as a control, repeated-dose radiotherapy, and DEVD-S-DOX/DCK complex (5 mg/kg) coupled with repeated-dose radiation over a period of 30 days.

FIG. 17D shows the tumors of HCC-70 xenografted mice that received saline as a control, repeated-dose radiotherapy, and DEVD-S-DOX/DCK complex (5 mg/kg) coupled with repeated-dose radiation over a period of 30 days.

FIG. 18 depicts a synthesis for preparing the DEVD-S-DCX/DCK complex.

FIG. 19 depicts the chemical structure of the DEVD-S-DCX/DCK complex.

FIG. 20A shows the tumor volume profile of human breast cancer MDA-MB-436 cells transplanted into female NSG SCID mice that received saline as a control, 3 mg/kg of DEVD-S-DCX/DCK (based on the docetaxel content) via daily oral administration, 50 mg/kg of olaparib via intraperitoneal injection daily for the first five days of every week for two weeks (5 days on/2 days off), or 3 mg/kg of DEVD-S-DCX/DCK (based on the docetaxel content) via daily oral administration in combination with 50 mg/kg of olaparib via intraperitoneal injection daily for the first five days of every week for two weeks (5 days on/2 days off).

FIG. 20B shows the body weight profile of human breast cancer MDA-MB-436 cells transplanted into female NSG SCID mice that received saline as a control, 3 mg/kg of DEVD-S-DCX/DCK (based on the docetaxel content) via daily oral administration, 50 mg/kg of olaparib via intraperitoneal injection daily for the first five days of every week for two weeks (5 days on/2 days off), or 3 mg/kg of DEVD-S-DCX/DCK (based on the docetaxel content) via daily oral administration in combination with 50 mg/kg of olaparib via intraperitoneal injection daily for the first five days of every week for two weeks (5 days on/2 days off).

### DETAILED DESCRIPTION

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described for illustrative purposes. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to ranges substantially around the number without departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular number.

As used herein, the term “tumor cell(s)” refers to the cells of any type of tumor tissue, benign or malignant.

As used herein, the term “cancer” refers to cancer originating from any part of the body or any cell type. This includes, but is not limited to, carcinoma, sarcoma, lymphoma, germ cell tumors, and blastoma. In some embodiments, the cancer is associated with a specific location in the body or a specific disease.

As used herein, the term “subject” refers to any animal in need of treatment by any one or more of the methods described herein, including humans and other mammals, such as dogs, cats, rabbit, horses, and cows. For example, a subject may be suffering from or at risk of developing a condition that can be treated or prevented with an apoptosis inducing treatment. In specific embodiments, the subject is a human with a tumor. In further specific embodiments, the tumor is malignant. In further specific embodiments, the subject is a human diagnosed with cancer.

Maximum tolerated dose (MTD) chemotherapy has often failed due to the complex nature of cancer and drug toxicity, suggesting that cancer should be treated as a chronic disease. Although metronomic therapy—a continuous and frequent administration of anticancer agents at minimally effective doses—using traditional cytotoxic agents display lower toxicity than its MTD equivalent, its efficacy is hindered by the limitations of intravenous administration. In one aspect, the complexes described herein can be used in radiotherapy-assisted orally available metronomic apoptosis-targeted chemotherapy, which offers increased therapeutic benefit via targeted chemotherapy can be conveniently carried out on a long-term basis with frequent administration. In another aspect, the complexes described herein can be used in combination with chemotherapy treatment, where the chemotherapy treatment induces apoptosis of cancer cells and the combination of metronomic dosing of the complexes and chemotherapy treatment provides a synergistic effect on tumor growth inhibition.

Described herein are complexes comprising an anticancer prodrug moiety that can selectively deliver an anticancer chemotherapeutic agent to tumor tissues and a bile acid moiety, where the bile acid moiety is complexed to the anticancer cancer agent. Provided in one aspect is a complex comprising: (a) an anticancer prodrug moiety comprising (i) a caspase-cleavable peptide, covalently attached directly or through a linker, to (ii) anticancer chemotherapeutic agent; and (b) a bile acid moiety, wherein the bile acid moiety is non-covalently complexed to the anticancer prodrug moiety. Such complexes can be used to target tumors and treat cancer, such as in methods for inducing apoptosis of tumor cells, including malignant (cancerous) tumor cells, including methods for inducing amplified apoptosis of tumor cells. In some embodiments, the methods include radiation-induced apoptosis-targeted chemotherapy (RIATC), including radiotherapy-assisted orally available metronomic apoptosis-targeted chemotherapy, which could be used long-term with very low adverse effects. In some embodiments, the methods include chemotherapy-induced apoptosis-targeted chemotherapy, including chemotherapy-assisted orally available metronomic apoptosis-targeted chemotherapy, which could be used long-term with very low adverse effects.

While not being bound by theory, it is believed that complexation of the bile acid moiety to the anticancer prodrug moiety allows for the intestinal absorption of the anticancer prodrug. After the complex is absorbed from the intestinal lumen into the blood vessel, the anticancer prodrug moiety is delivered to tumor tissue in which apoptosis has been induced by artificial stimuli (e.g., radiation therapy). Upon reaching tumor tissue in which apoptosis has been induced, caspase expressed (secreted) by apoptotic cells selectively cleaves the anticancer drug from the anticancer prodrug moiety and thereby selectively releases the drug at the tumor site. As demonstrated in the Examples, complexes described herein provide a significant anticancer effect, such as suppression of tumor effect.

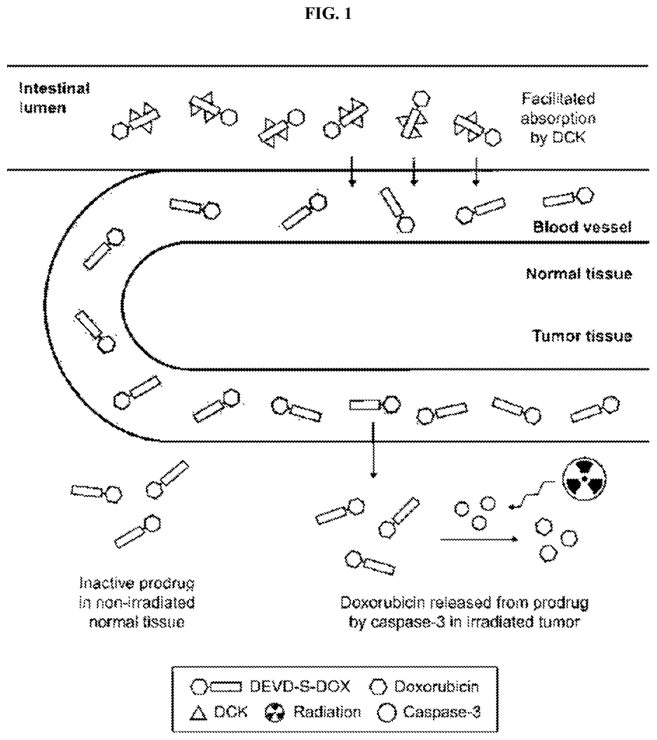

For example, the anticancer prodrug moiety DEVD-S-DOX was electrostatically complexed with the bile acid moiety DCK (N^{α}-deoxycholic-L-lysine-methylester) for oral administration, gastrointestinal absorption, and delivery to irradiated tumor tissue. Once delivered to irradiated tumor tissue, the doxorubicin is released via proteolytic activity of upregulated caspase, such as caspase-3, expressed by apoptotic cells. A schematic illustration of this example of radiotherapy-assisted orally available metronomic apoptosis-target therapy is depicted in FIG. 1.

In another example, the anticancer prodrug moiety DEVD-S-DCX was electrostatically complexed with the bile acid moiety DCK (N^{α}-deoxycholic-L-lysine-methylester) for oral administration, gastrointestinal absorption, and delivery to tumor tissue subjected to targeted chemotherapy (e.g. olaparib for BRCA mutant cancer) to induce apoptosis. Once delivered to the tumor tissue, the docetaxel is released via proteolytic activity of upregulated caspase, such as caspase-3, expressed by apoptotic cells initially induced by the initial chemotherapeutic agent (e.g. olaparib).

In some embodiments, any of the complexes described herein may further comprise a dye. In such embodiments, the dye may be conjugated to any suitable part of the anticancer prodrug moiety, including the caspase-cleavable peptide or anticancer chemotherapeutic agent.

### Anticancer Complex

As noted above, the complexes described herein comprise (a) an anticancer prodrug moiety comprising (i) a caspase-cleavable peptide, covalently attached directly or through a linker, to (ii) an anticancer chemotherapeutic agent; and (b) a bile acid moiety, wherein the bile acid moiety is non-covalently complexed to the anticancer prodrug moiety.

In some embodiments, a plurality of bile acid moieties is covalently or non-covalently complexed to a single anticancer prodrug moiety. In some embodiments, one bile acid moiety is non-covalently complexed to a single anticancer prodrug moiety. In some embodiments, two or more bile acid moieties are non-covalently complexed to a single anticancer prodrug moiety. In some embodiments, three or more bile acid moieties are or non-covalently complexed to a single anticancer prodrug moiety. In some embodiments, one, two, or three, or more bile acid moieties are non-covalently complexed to a single anticancer prodrug moiety.

In some embodiments, the complex comprises: (a) an anticancer prodrug moiety comprising (i) a caspase-cleavable peptide comprising the amino acid sequence Asp-Glu-Val-Asp joined through a para-aminobenzyloxycarbonyl linker to (ii) an anticancer chemotherapeutic agent; and (b) a bile acid moiety comprising Na-deoxycholyl-L-lysine-methylester. In some embodiments, the anticancer chemotherapeutic agent is doxorubicin. In some embodiments, the anticancer chemotherapeutic agent is docetaxel.

### Anticancer Prodrug Moieties

The anticancer prodrug moieties described herein comprise (i) a caspase-cleavable peptide, covalently attached directly or through a linker, to (ii) an anticancer chemotherapeutic agent, as described in more detail below. Suitable anticancer prodrug moieties also include those disclosed in U.S. Pat. Nos. 9,408,910 and 9,408,911, which are incorporated herein by reference in their entirety, including their disclosures of such compounds and how to make them.

### Caspase-Cleavable Peptide Linker

As noted above, the anticancer prodrug moiety described herein includes a caspase-cleavable peptide linker, wherein the caspase-cleavable peptide is cleavable by caspase.

As used herein, the term “caspase” refers to cysteine-aspartic proteases and cysteine-dependent aspartate-directed proteases that are activated (e.g., expressed) by cells undergoing apoptosis. In specific embodiments, the caspase is caspase-3, caspase-7, and/or caspase-9.

As used herein, the term “amino acid” refers to any amino acid, including naturally-occurring amino acids and non-naturally-occurring amino acids, including synthetically made naturally-occurring amino acids. The natural amino acids, with exception of glycine, contain a chiral carbon atom. Thus, amino acids can be in the form of an L or D isomer. Specific examples of amino acids include β-alanine (BALA), γ-aminobutyric acid (GABA), 5-aminovaleric acid, glycine (Gly or G), phenylglycine, arginine (Arg or R), homoarginine (Har or hR), alanine (Ala or A), valine (Val or V), norvaline, leucine (Leu or L), norleucine (Nle), isoleucine (Ile or I), serine (Ser or S), isoserine, homoserine (Hse), threonine (Thr or T), allothreonine, methionine (Met or M), ethionine, glutamic acid (Glu or E), aspartic acid (Asp or D), asparagine (Asn or N), cysteine (Cys or C), cystine, phenylalanine, tyrosine (Tyr or Y), tryptophan (Trp or W), lysine (Lys or K), hydroxylysine (Hyl), histidine (His or H), ornithine (Orn), glutamine (Gln or Q), citrulline, proline (Pro or P), and 4-hydroxyproline (Hyp or O).

As used herein, the term “peptide” refers to peptides and peptide analogs, wherein peptide analogs may include naturally-occurring amino acids and non-naturally-occurring amino acids, modifications such as glycosylations, modified R groups, and/or modified peptide backbones. In some embodiments, a peptide comprises only L-isomers of its chiral amino acids. In other embodiments, a peptide comprises only D-isomers of its chiral amino acids. In other embodiments, a peptide comprises both L-isomers and D-isomers of one or more of its chiral amino acids. The term “peptide” also includes peptides or peptide analogs that include amino acid mimetics that function in a manner similar to the naturally-occurring amino acids. In specific embodiments, peptide analogs include at least one bond in the peptide sequence which is different from an amide bond, such as urethane, urea, ester or thioester bond. Peptides or peptide analogs as used herein can be linear, cyclic or branched, but typically are linear.

As used herein, the term “caspase-cleavable peptide linker” refers to a peptide sequence of two or more one amino acid residues that is capable of being cleaved by caspase. In some embodiments, the caspase cleavable peptide linker is cleavable by caspase-3 or caspase-7, such as peptides comprising the sequence Asp-Xaa-Xaa-Asp (SEQ ID NO:1) (where “Xaa” represents any amino acid, in L- or D-isomer form). In some embodiments, the caspase-cleavable peptide linker is cleavable by caspase-9, such as peptides comprising the amino acid sequence Leu-Xaa-Xaa-Asp (SEQ ID NO:2) or Val-Xaa-Xaa-Asp (SEQ ID NO:3) (where “Xaa” represents any amino acid, in L- or D-isomer form).

In specific embodiments, the caspase-cleavable peptide linker comprises one of the following sequences:

(SEQ ID NO: 4)
Asp-Glu-Val-Asp
(SEQ ID NO: 5)
Asp-Leu-Val-Asp
(SEQ ID NO: 6)
Asp-Glu-Ile-Asp,
or
(SEQ ID NO: 7)
Leu-Glu-His-Asp.

In specific embodiments, the caspase-cleavable peptide linker comprises the sequence Asp-Glu-Val-Asp (SEQ ID NO:4), also denoted as DEVD.

In specific embodiments, the caspase-cleavable peptide linker comprises the sequence Lys-Gly-Asp-Glu-Val-Asp (SEQ ID NO:8), also denoted as KGDEVD.

In some embodiments, the caspase-cleavable peptide may include the amino acid sequence Asp-Glu-Val-Asp, wherein an anticancer chemotherapeutic agent is conjugated to the C-terminus. Upon cleavage by caspase through hydrolysis, the anticancer chemotherapeutic agent is separated from the peptide and released into its active form, such that it exhibits its anticancer effect.

In certain embodiments, the anticancer prodrug moiety is inactive in the conjugate form, until it is cleaved from the conjugate, such as by caspase cleavage of the caspase-cleavable peptide. In this context, “inactive” refers to activity that is at least 100-fold less cytotoxic than the active form. In accordance with such embodiments, the anticancer prodrug moiety exerts minimal damage to healthy cells, because it only is activated in the presence of caspase, e.g., in the presence of cells undergoing apoptosis, such as tumor cells undergoing apoptosis, such as tumor cells in which apoptosis was induced by artificial stimuli (e.g., radiation therapy). Thus, in certain embodiments, the complex described herein exhibits minimal side effects because the chemotherapeutic agent is not active until it is released from the conjugate at the target site.

### Anticancer Chemotherapeutic Agent

As noted above, the anticancer prodrug moiety described herein includes an anticancer chemotherapeutic agent.

As used herein, the term “anticancer chemotherapeutic agent” refers to a moiety useful to treat cancer, such as a small molecule chemical compound used to treat cancer. In specific embodiments, the anticancer chemotherapeutic agent induces apoptosis in target cells, e.g., in tumor cells and tumor tissue. Any anticancer chemotherapeutic agent can be used as an anticancer chemotherapeutic agent in the prodrug moieties described herein.

In some embodiments, the anticancer chemotherapeutic agent is selected from anthracyclines, antibiotics, alkylating agents, platinum-based agents, antimetabolites, topoisomerase inhibitors, and mitotic inhibitors. In some embodiments, the anticancer chemotherapeutic agent is an anthracycline, such as doxorubicin, daunorubicin, epirubicin, idarubicin, valrubicin, or a derivative thereof; an antibiotic, such as actinomycin-D, bleomycin, mitomycin-C, calicheamicin, or a derivative thereof; an alkylating agent, such as cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, or a derivative thereof; a platinum-based agent, such as cisplatin, carboplatin, nedaplatin, oxaliplatin, satraplatin, triplatin tetranitrate, or a derivative thereof; an antimetabolite, such as 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, or a derivative thereof; a topoisomerase inhibitor, such as camptothecin, topotecan, irinotecan, etoposide, teniposide, mitoxantrone, or a derivative thereof; a mitotic inhibitor, such as paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, or a derivative thereof. In some embodiments, the chemotherapeutic agent is a PARP inhibitor, which include but are not limited, to olaparib, rucaparib, niraparib, talazoparib, veliparib, and iniparib. In some embodiments, the chemotherapeutic agent is trastzumab, ado-trastuzumab emtansine, tamoxifen, lapatinib, palbociclib, ribociclib, neratinib maleate, or abemaciclib. In specific embodiments, the anticancer chemotherapeutic agent is doxorubicin or daunorubicin. In specific embodiments, the anticancer chemotherapeutic agent is doxorubicin. In specific embodiments, the anticancer chemotherapeutic agent is docetaxel.

### Linkages and Linkers Conjugates

In some embodiments the caspase-cleavable peptide linker is conjugated directly to the anticancer chemotherapeutic agent, such as by a covalent bond between a moiety at the C-terminus or N-terminus of the peptide or on a side chain of the peptide and a moiety on the chemotherapeutic agent.

In some embodiments the caspase-cleavable peptide linker is conjugated to the anticancer chemotherapeutic agent through a linker. Any linker suitable for use in pharmaceutical compounds may be used. Suitable linkers include para-aminobenzyloxycarbonyl (PABC), aminoethyl-N-methylcarbonyl, aminobiphenylmethyloxycarbonyl, a dendritic linker and a cephalosporin-based linker. Other suitable linkers include ethylenediamino acid linkers and dipeptide-based linkers (e.g., H-Ser-Pro-OH ester linkages). Suitable linkers are illustrated in the examples, including PABC.

### Bile Acid Moiety

As noted above, the complex comprises a bile acid moiety non-covalently complexed to the anticancer prodrug moiety. The bile acid moiety can be a bile acid or bile acid derivative. Without being bound by theory, the bile acid moiety can function as an oral absorption enhancer to improve the bioavailability of the orally administered complex. In some embodiments, the bile acid moiety promotes intestinal absorption of the anticancer prodrug moiety. Suitable bile acid moieties include the bile acids and bile acid derivatives described in U.S. Pat. No. 7,906,137, which is incorporated herein by reference in its entirety, including its disclosure of such compounds.

In some embodiments, the bile acid moiety is selected from one or more of the following: cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, and hyodeoxycholic acid. In some embodiments, the bile acid moiety is a derivative of deoxycholic acid, such as a positively charged derivative of deoxycholic acid. In some embodiments, the bile acid moiety is Na-deoxycholyl-L-lysine-methylester.

Suitable bile acid derivatives include positively charged bile acid derivatives, such as deoxycholic amine. Suitable bile acid derivatives can be prepared, for example, by conjugating a bile acid with a small moiety, such as a moiety that will confer a positive charge, such as a primary amine moiety, such as an ethylene amine moiety, or an amino acid. In some embodiments, the bile acid moiety includes a bile acid conjugated to an amino acid.

In some embodiments, the bile acid moiety comprises an anionic (e.g., carboxylic group) or a cationic group (e.g., primary amine group), which may form an ionic bond with the prodrug moiety. In some embodiments, the bile acid moiety, such as Na-deoxycholyl-L-lysine-methylester, comprises a primary amine group.

In some embodiments, the bile acid moiety is non-covalently complexed to the anticancer prodrug moiety by ionic bonding, hydrophobic bonding or coordinate bonding, where ionic bonding may be preferred. In some embodiments, the bile acid moiety is complexed by an ionic bond at a positive or negative ionic region of the caspase-cleavable peptide or anticancer chemotherapeutic agent. In some embodiments, the bile acid moiety is complexed by an ionic bond to a carboxylic group of the caspase-cleavable peptide, or to a carboxylic group of the anticancer chemotherapeutic agent.

In some embodiments, the complexation of the bile acid moiety to the anticancer prodrug moiety increases hydrophobicity through ionic bonding of the carboxylic group of the caspase-cleavable peptide or anticancer chemotherapeutic agent. In some embodiments, the complexation enhances gastrointestinal absorption through interaction with bile acid transporters of the small intestine, thus increasing bioavailability of the drug when orally administered. In some embodiments, gastrointestinal absorption involves absorption by bile acid transporters in the small intestine. In some embodiments, gastrointestinal absorption involves diffusion through gastrointestinal membrane attributed to increased hydrophobicity imparted from complexing the anticancer prodrug moiety with a bile acid moiety.

### Complex Preparation

The complexes described herein are prepared by complexing an anticancer prodrug moiety as described herein to a bile acid moiety as described herein.

An anticancer prodrug moiety as described herein can be made by any suitable method, including as disclosed in U.S. Pat. Nos. 9,408,910 and 9,408,911. In some embodiments, an anticancer prodrug moiety is prepared as follows: (i) reaction of the C-terminus of the caspase cleavable peptide, whose side chain is protected, with para-aminobenzylalcohol to provide a hydroxyl group; and (ii) reaction of the resulting hydroxyl group with nitrophenyl carbonate followed by the reaction with the anticancer chemotherapeutic agent to form a carbamate.

In some embodiments, the bile acid moiety is added to a solution or suspension comprising the anticancer prodrug moiety, such as at a pH of about 7, such as with stirring. The complex forms as a precipitate, which can be isolated by centrifugation and/or filtration.

### Compositions

In some embodiments provided herein are pharmaceutical compositions comprising the complexes described herein and a pharmaceutically acceptable carrier, excipient, and/or diluent. Examples of suitable carriers, excipients, and diluents include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, minerals, and the like.

The pharmaceutical composition may be prepared for any route of administration, including any parenteral or local route of administration. In some embodiments, the pharmaceutical composition is suitable for injection or infusion, such as for intravenous injection or infusion, such as being prepared as a sterile composition for injection or infusion. In other embodiments, the pharmaceutical composition is suitable for inhalation, such as being in the form of a nasal or oral spray or aerosol. In other embodiments, the pharmaceutical composition is suitable for rectal or vaginal administration, such as being in a suppository formulation. In other embodiments, the pharmaceutical composition is suitable for topical or transdermal administration, such as being in a solution, emulsion, gel, or patch. In particular embodiments, the pharmaceutical composition is formulated for oral administration, such as being prepared in a powder, granule, tablet, capsule, suspension, emulsion, or syrup form. Appropriate components and excipients for such compositions are known in the art.

In some embodiments, the pharmaceutical compositions described herein are formulated for oral administration. In some embodiments, the pharmaceutical compositions are manufactured in the form of powder, granule, tablet, capsule, suspension, emulsion or syrup. In some embodiments, the pharmaceutical compositions are manufactured in the form of oral spray or aerosol to be suitable for inhalation. Any diluting agents suitable for such compositions may be used.

Examples of solid formulations for oral administration include a tablet, a pill, a powder, a granule, a capsule, or the like. Solid formulations may be manufactured by mixing the complexes described herein with one or more diluting agents, such as starch, calcium carbonate, and lactose gelatin. Additionally, a lubricant, such as magnesium stearate or talc, may be added to aid tableting or other processes.

Examples of a liquid medication for oral administration include a solution, a suspension, an emulsion, a syrup, and the like, include aqueous (water-based) liquids. In some embodiments, the liquid may include one or more additives, such as wetting agents, sweetening agents, flavoring agents, preservatives, and the like, and optionally, liquid paraffin.

In specific embodiments, the complex is dissolved in water or another pharmaceutically acceptable aqueous carrier in which the complex exhibits good solubility, optionally with or without other pharmaceutical acceptable excipients, preservatives, and the like.

### Therapeutic Methods Using Complexes

As noted above, the complexes described herein are useful in methods of inducing apoptosis, including amplifying apoptosis, such as apoptosis of tumor cells, including malignant tumor cells, including methods of treating cancer, including radiation-induced apoptosis-targeted chemotherapy (RIATC), including radiotherapy-assisted orally available metronomic apoptosis-targeted chemotherapy. In some embodiments, the complex is administered to a subject in need of treatment of cancer.

In some embodiments, the subject is subjected to a treatment that induces apoptosis in target cells, thereby inducing expression of caspase, prior to or concurrently with administration of the complex. In accordance with such some embodiments, apoptosis may be induced by any therapeutically acceptable means, such as a treatment selected from one or more of radiation therapy, hyperthermia, laser therapy, photodynamic therapy, chemotherapy, and cryosurgery, or targeted therapy, such as a small molecule tyrosine kinase inhibitor (TKI) or monoclonal antibody that targets tumor cells. In some embodiments, the treatment is radiation therapy (or radiotherapy). In some embodiments, the treatment is chemotherapy, in which case the chemotherapeutic agent for inducing apoptosis may be any chemotherapeutic agent, including those disclosed above, and may be the same as or different from the anticancer chemotherapeutic agent of the anticancer prodrug moiety. In some embodiments, the chemotherapeutic agent for inducing apoptosis is a PARP inhibitor, such as olaparib, rucaparib, niraparib, talazoparib, veliparib, and iniparib. In some embodiments, the chemotherapeutic agent for inducing apoptosis is trastzumab, ado-trastuzumab emtansine, tamoxifen, lapatinib, palbociclib, ribociclib, neratinib maleate, or abemaciclib. In specific embodiments, the chemotherapeutic agent for inducing apoptosis is olaparib. In specific embodiments, including where the tumor or cancer has metastasized and/or is unidentifiable, the apoptotic inducing treatment may include targeted therapy, such as TKIs, antibodies, aptamers, or targeted nanoparticles, which target tumor cells.

In specific embodiments, which may be referred to as radiation-induced apoptosis-targeted chemotherapy (RIATC), apoptosis is induced by radiation therapy. As used herein, the term “radiation therapy” refers to all methods of radiation therapy, including external beam radiation therapy, sealed source ration therapy, and systemic radioisotope therapy. In some embodiments, the radiation is focused locally to the target site, such as to a tumor site. In some embodiments, radiation therapy is given prior to administration of the complex. In any embodiments using radiation therapy, the radiation therapy may include gamma-knife radiation, cyber-knife radiation, and/or high intensity focused ultrasound radiation.

In some embodiments, the radiation therapy involves treatment with a low dose of radiation. In specific embodiments, an adult human subject is treated with radiation at a dose of up to about 70 Gy. In specific embodiments, an adult human subject is treated with radiation at a dose of from 1 to 35 Gy. In other embodiments, an adult human subject is treated with a single dose of radiation of up to about 35 Gy. In other embodiments, an adult human subject is treated with radiation at weekly doses of up to about 10 Gy. In other embodiments, an adult human subject is treated with radiation at weekly doses of up to about 35 Gy. In other embodiments, an adult human subject is treated with weekly administration of radiotherapy at a dose of 1 to 35 Gy. In some embodiments, an adult human subject is treated with weekly administration of radiotherapy at a dose of 2 to 10 Gy. In some embodiments, an adult human subject is treated with weekly administration of radiotherapy at a dose of 2, 3, 4, 5, 6, 7, 8, 9 or 10 Gy.

As noted above, in some embodiments, which may be referred to as chemotherapy-induced apoptosis-targeted chemotherapy, apoptosis is initially induced by a chemotherapeutic agent. The chemotherapeutic agent for inducing apoptosis may be any chemotherapeutic agent, such as any one of those disclosed herein, and may be the same as or different from the anticancer chemotherapeutic agent of the anticancer prodrug moiety. In some embodiments, the chemotherapeutic agent for inducing apoptosis is specific for the type of cancer and/or tumor being targeted, such as BRCA mutant cancers (e.g. BRCA 1 and BRCA 2). In some embodiments, the chemotherapeutic agent for inducing apoptosis is a PARP inhibitor, as discussed above.

In some embodiments, the chemotherapeutic agent for inducing apoptosis involves treatment with a low dose, such as a dose lower than the dose that would be used if the chemotherapeutic agent was the only agent being administered. In some embodiments, the dose administered to a subject may be between about 1 mg/kg and about 200 mg/kg, or between about 1 mg/kg and 100 mg/kg, including from about 25 mg/kg to about 75 mg/kg, such as from about 10 mg/kg to about 50 mg/kg, or greater. In some embodiments, the chemotherapeutic agent is administered once, twice, three, four, or five times a day. In some embodiments, the chemotherapeutic agent is administered once, twice, three, four, five, six, or seven times a week. In some embodiments, the chemotherapeutic agent is administered once, twice, three, four, five, six, or seven times every two weeks.

In specific embodiments, the methods described herein induce and, optionally, amplify, apoptosis by the following process: Apoptosis is induced in cells at the target site by a low dose of radiation or administration of a chemotherapeutic agent, as disclosed above, resulting in expression of caspase. The complex comprising the anticancer prodrug moiety and the bile acid moiety is orally administered, and the anticancer prodrug moiety is absorbed from the intestines and delivered to the target site. The caspase-cleavable peptide linker is cleaved by caspase expressed by apoptotic cells at the target site, releasing the anticancer chemotherapeutic agent at the target site. The anticancer chemotherapeutic agent induces apoptosis of additional cells, resulting in additional expression of caspase, resulting in further caspase-induced cleavage of additional anticancer prodrug moiety, inducing apoptosis of additional cells, resulting in amplified apoptosis. This amplification yields methods with high efficiency and specificity in killing target cells, such as target tumor cells. Moreover, this amplification effect can permit a longer time interval between doses of radiation or chemotherapeutic agent and/or between administrations of doses of the prodrug conjugate. Thus, in some embodiments, this amplification effect may reduce the amount of anticancer chemotherapeutic agent and/or radiation needed to treat a certain number of cancer cells.

The dose of complex administered will vary depending on the subject and the condition for which it is administered, and can be determined by someone of skill in the art. In some embodiments, the dose administered to a subject may be between about 1 mg/kg and about 100 mg/kg, including from about 5 mg/kg to about 75 mg/kg, such as from about 10 mg/kg to about 50 mg/kg, or greater. In some embodiments, the dose administered to a subject may be between about 1 mg/kg and about 100 mg/kg, including from about 1 mg/kg to about 50 mg/kg, such as from about 1 mg/kg to about 20 mg/kg, or greater. In specific embodiments, the complex exhibits lower toxicity than the chemotherapeutic agent alone, such that the dose administered may be higher than that which would be non-toxic for the chemotherapeutic agent alone. In some embodiments, daily administration of the complex at a dose of 1 to 10 mg/kg, based on the molar equivalent dose of the anticancer chemotherapeutic agent.

In some embodiments the subject is treated with a further apoptosis inducing treatment after the conjugate is administered. In such embodiments, the subsequent apoptosis inducing treatment may be the same as or identical to the previous apoptosis inducing treatment. Alternatively, the subsequent apoptosis inducing treatment may be different from the previous apoptosis inducing treatment. Possible differences include, but are not limited to, the type of treatment (e.g. radiation therapy, hyperthermia, laser therapy, photodynamic therapy, chemotherapy, cryosurgery, or targeted therapy), the anticancer chemotherapeutic agent or molecule for targeted therapy used; the radiation therapy used, and/or the dosage or duration of treatment, and any other variation of an apoptosis inducing treatment.

In some embodiments, the method comprises weekly administration of radiotherapy, such as at a dose of 1 to 35 Gy, and daily administration of the complex, such as at a dose of 1 to 100 mg/kg, based on the molar equivalent dose of the anticancer chemotherapeutic agent. In some embodiments, the method comprises weekly administration of radiotherapy at a dose of 2 to 10 Gy, and daily administration of the complex at a dose of 1 to 10 mg/kg, based on the molar equivalent dose of the anticancer chemotherapeutic agent. In some embodiments, the method results in gastrointestinal absorption of the chemotherapeutic prodrug

As noted above, the anticancer prodrug moiety is inactive prior to cleavage from the caspase-cleavable peptide linker. Thus, the anticancer prodrug moiety is not toxic (or apoptotic) to healthy cells. In specific embodiments, the methods described herein reduce damage to normal cells by about 10%, 20%, 30%, 40%, 50%, 60%, 70%, 80% or more, as compared with administration of the same anticancer chemotherapeutic agent in non-conjugated form.

Moreover, the apoptotic effect of the anticancer prodrug moiety is selective to cells expressing caspase, e.g., cells undergoing apoptosis. Thus, once apoptosis is induced in a region of target cells (e.g., in target tissue), the methods described herein selectively and effectively induce apoptosis of other target cells, thereby, for example, treating cancer. In some embodiments, the delivery of anticancer chemotherapeutic agents is effective regardless of the genotype of cancer tissue.

### EXAMPLES

The following specific examples are included as illustrative of the compositions described herein. These examples are in no way intended to limit the scope of the disclosure. Other aspects of the disclosure will be apparent to those skilled in the art to which the disclosure pertains.

### Example 1—Synthesis of DEVD-S-DOX/DCK

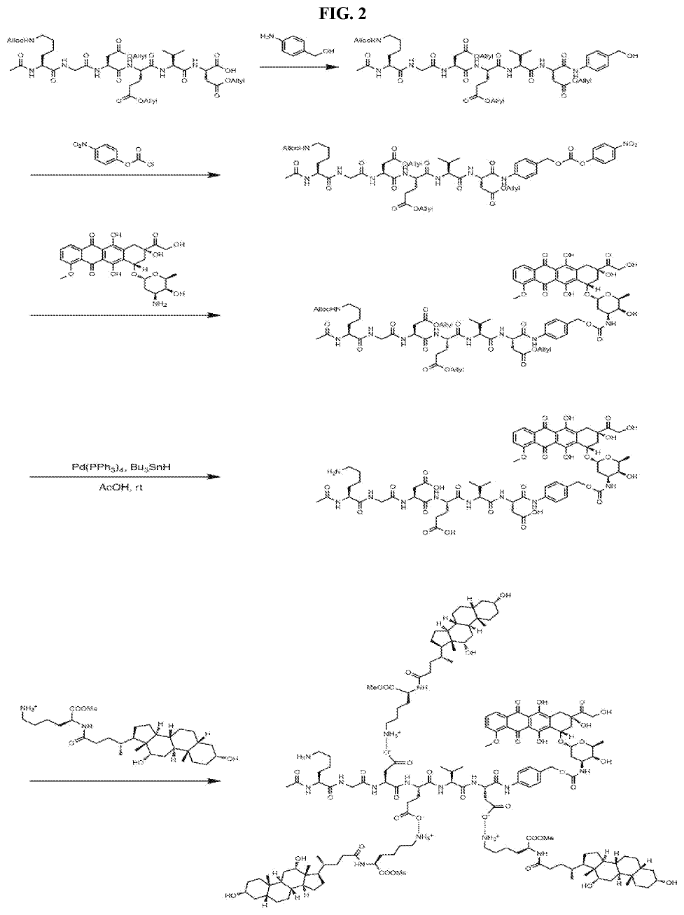

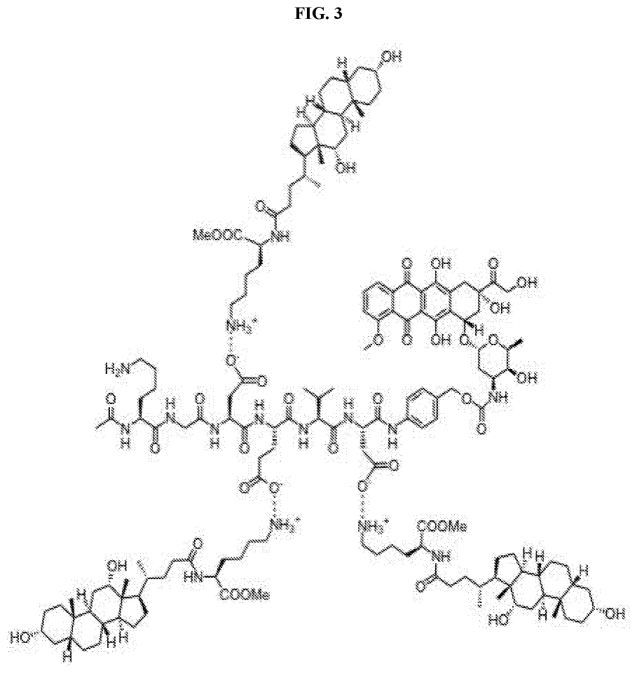

The DEVD-S-DOX/DCK complex was prepared as outlined below and contained doxorubicin as the anticancer chemotherapeutic agent covalently linked at the C-terminus of Ac-KGDEVD peptide (DEVD-S-DOX). Complexation with N-deoxycholyl-L-lysine-methyl ester (DCK), a bile acid moiety, through ionic bonding, formed the DEVD-S-DOX/DCK complex. This complex can be orally administered to deliver the anticancer prodrug moiety (DEVD-S-DOX) through intestinal absorption to cancer tissue. Radiation therapy, or any suitable external stimuli, may be used to induce apoptosis in the cancer tissue, leading to caspase expression. Hydrolysis of the peptide sequence of Asp-Glu-Val-Asp releases doxorubicin which exerts its anticancer effect. FIG. 2 shows the synthesis scheme described below for the preparation of the DEVD-S-DOX/DCK complex and FIG. 3 shows the structure of the DEVD-S-DOX/DCK complex.

Ac-K(OAlloc)GD(OAll)E(OAll)VD(OAll)-OH (344 mg, 0.38 mmol), 4 aminobenzyl alcohol (2 molar equivalents), and EEDQ (2 molar equivalents) were dissolved in anhydrous DMF (11 mL), followed by stirring under argon gas at room temperature for 24 hours. After vacuum evaporation, the solution was crystalized in ether to obtain K(OAlloc)GD(OAll)E(OAll)VD(OAll)-PABOH.

AcK(OAlloc)GD(OAll)E(OAll)VD(OAll)-PABOH (322 mg, 0.318 mmol) and 4-nitrophenyl chloroformate (1.2 molar equivalents) were dissolved in anhydrous CH2Cl2(10 mL), followed by stirring in of 2,6-lutidine (3 molar equivalents) at room temperature. After 2 hours, anhydrous DMF (2 mL) was added to the reactant, followed by the addition of 2,6-lutidine (2 molar equivalents). After 24, 27, and 46 hours, each of 2,6-lutidine (4.75 molar equivalents) and 4-nitrophenyl chloroformate (1 molar equivalent) were stirred in at room temperature, followed by addition of sodium bicarbonate solution at 84 hours to complete the reaction. The reactant was extracted three times by ethyl acetate, and the organic layer was washed using 0.5 M citric acid and salt water. The obtained organic layer was passed through a sodium sulfate layer to remove the remaining water, and the solvent was removed by decompression, followed by addition of ether to crystallize it. The obtained product was purified by preparative HPLC (77 mg, 20.5%). ESI-MS: m/z 1200.54 [M+Na]+.

The obtained Ac-K(OAlloc)GD(OAll)E(OAll)VD(OAll)-PABC (77 mg, 0.065 mmol) and Doxorubicin HCl (1.2 molar equivalents) were dissolved in anhydrous DMF (8 mL), followed by the addition of DIEA (5.4 molar equivalents) and stirring at room temperature for 16 hours. After removing the solvent by decompression, Ac-K(OAlloc)GD(OAll)E(OAll)VD(OAll)-PABC-Doxorubicin was separated using preparative HPLC and obtained as a solid (red non-crystalline solid, 76 mg, 74%). ESI-MS: m/z 1605.06 [M+Na]+.

Ac-K(OAlloc)GD(OAll)E(OAll)VD(OAll)-PABC-Doxorubicin (50 mg, 0.032 mmol) and Pd(PPh₃)₄ (0.2 molar equivalents) were dissolved in anhydrous DMF (7.6 mL), followed by the addition of acetate acid (20 molar equivalents) and tributyltin hydride (17.3 molar equivalents) to the reactant, stirring for 1 hour at room temperature to provide the peptide residual with deprotection. The solvent of the reacting solution was removed under decompression and purified by preparative HPLC to be obtained as DEVD-S-DOX (Ac-KGDEVD-PABC-Doxorubicin, (red non-crystalline solid, 6 mg, 13.6%). ESI-MS: m/z 1378.4 [M+H]+.

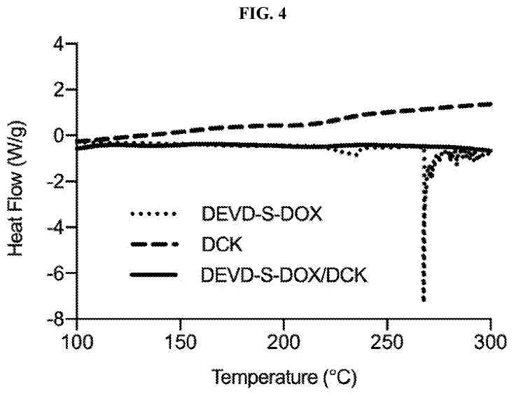

The obtained DEVD-S-DOX (6 mg, 0.004 mmol) and DCK (12 mg, 0.022 mmol, 5 molar equivalents) were dissolved in distilled water (1 mL), followed by adding 0.1 N NaHCO₃ solution to set the solution at pH 7. The obtained solution was filtered, washed three times in distilled water (5 mL), and decompressed to obtain as DEVD-SDOX/DCK complex (red non-crystalline solid 11.5 mg, 89%). The loss of crystallinity in the obtained complex was confirmed by the differential scanning calorimeter, which confirmed that the characteristic endothermic peak of DEVD-S-DOX has disappeared at 267.64° C. (FIG. 4).

### Example 2—Assessment of Cytotoxicity of DEVD-S-DOX

The cytotoxicity of DEVD-S-DOX in human breast cancer cells MDA-MB-231 was assessed using MTT assay with or without the treatment with human recombinant caspase-3. After the cancer cells were placed on a 96-well microplate at the concentration of 5×10⁴ cell/well and cultured in DMEM (10% FBS) media for 24 hours, doxorubicin, DEVD-S-DOX, and DEVD-S-DOX, pre-treated with caspase-3 (500 ng/mL), were added at concentrations ranging from 0.01-100 μM, and further cultured for 48 hours. After that, 10 μL of MTT agent were added to each well and cultured for 2 hours at 37° C. until a blue precipitate forms. At this point, 100 μL of detergent agent was added and cultured for 4 hours at room temperature. Optical density was measured in each well at 570 nm to determine cell viability.

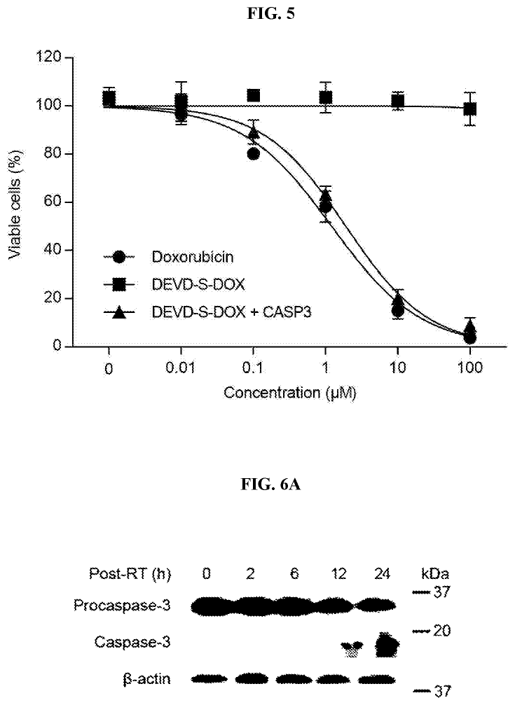

FIG. 5 depicts the results and shows that DEVD-S-DOX in the drug treated MDA-MB-231 cancer cells showed no cytotoxicity up to the point of 100 μM, which is the maximum concentration of the drug. On the other hand, when pretreated with caspase-3, DEVD-S-DOX showed a similar level of cytotoxicity as doxorubicin (DEVD-S-DOX: IC₅₀=1.78 μM; doxorubicin: IC₅₀=1.27 confirming that the cytotoxicity of DEVD-S-DOX was activated by caspase-3.

### Example 3—In-Vitro Assessment of Combined Effect of DEVD-S-DOX and Radiotherapy

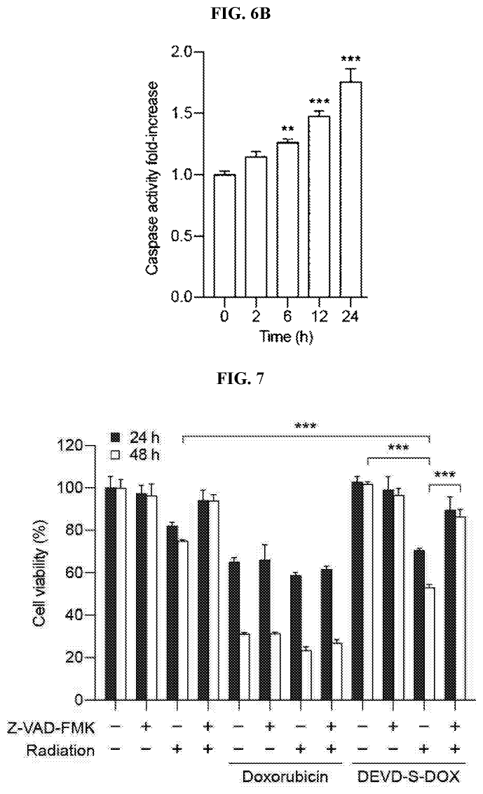

The degree of cytotoxicity of DEVD-S-DOX was assessed using an in vitro MTT assay and combined radiation therapy in the MDA-MB-231 human breast cancer cell line. The MDA-MB-231 cell line was irradiated with radiation and the expression of caspase-3 was evaluated. Western blot (FIG. 6A) and a cellular caspase-3 activity assay (FIG. 6B) confirmed that the expression of caspase-3 increased over time. For the Western blot, a detectable amount of caspase-3 was observed after 12 hours of irradiation and was significantly increased after 24 hours. Consistently, the cellular caspase-3 activity assay also showed a gradual increase of caspase-3 activity over time after the irradiation.

Next, the cancer cells were cultured in a 96-well microplate at the density of 5×10⁴ cell/well in DMEM (10% FBS) medium for 24 hours, and then treated with DEVD-S-DOX or 5 μM Doxorubicin. Some of the test groups were also treated with Z-VAD-FMK, which is a caspase inhibitor. Then cells were irradiated with 4 Gy of radiation, and after 24 and 48 hours, cell viability was assessed against the controls using an MTT assay (FIG. 7). The upregulation of caspase-3 from MDA-MB-231 cells after irradiation resulted in significant induction of cytotoxic activity of DEVD-S-DOX only when coupled with radiotherapy. While treatment with DEVD-S-DOX accompanied with radiotherapy inhibited the mean cell viability to 52.86%, the DEVD-D-DOX by itself did not show any noticeable cytotoxicity. The radiotherapy alone caused some degree of cell growth inhibition (mean cell viability: 74.91%), but was significantly weaker than the combinatory treatment (p<0.001). The combinatory effect was not observed when the cells were also treated with a caspase inhibitor (Z-VAD-FMK), indicating that the caspase-3 upregulated by the radiotherapy has indeed activated the DEVD-S-DOX which in turn induced cell death. This confirmed that caspase-3 was expressed via radiation therapy in the cancer cells, which then activated DEVD-S-DOX and led to a synergistic cytotoxic effect.

### Example 4—Penetration Rate of DEVD-S-DOX in the Cell Nucleus Depending on the Use of Radiation Therapy

The distribution of DEVD-S-DOX within the cell depending on the use of radiation therapy was observed in a human breast cancer cell line MDA-MB-231 through the florescent microscope. The cancer cells were cultured in a cell culture dish that is observable with a microscope, then treated with DEVD-S-DOX or Doxorubicin at a concentration of 5 μM. Some of the test groups were treated with Z-VAD-FMK, a caspase inhibitor. After that, the cancer cells were irradiated with 4Gy and cultured for an additional 24 hours. The cancer cells were fixed with formalin and the nucleus was dyed with DAPI, and then the cancer cells were observed with a florescent microscope. FIG. 8 depicts these results. Observation showed that when treated only with DEVD-S-DOX, the drug was distributed throughout the cytoplasm, but when treated along with radiation therapy, the drug was distributed in the nucleus, the cell organelle that the anticancer chemotherapeutic agent targets. Treatment with caspase inhibitor inhibited this effect, as movement of drug distribution within the cell with radiation therapy was not identified with caspase inhibitor treatment. As such, it was confirmed that DEVD-S-DOX was hydrolyzed by caspase-3, which is induced by radiation therapy, and resulted in active drug accumulation in the nucleus, showing cytotoxicity.

### Example 5—Pharmacokinetic Evaluation of DEVD-S-DOX/DCK

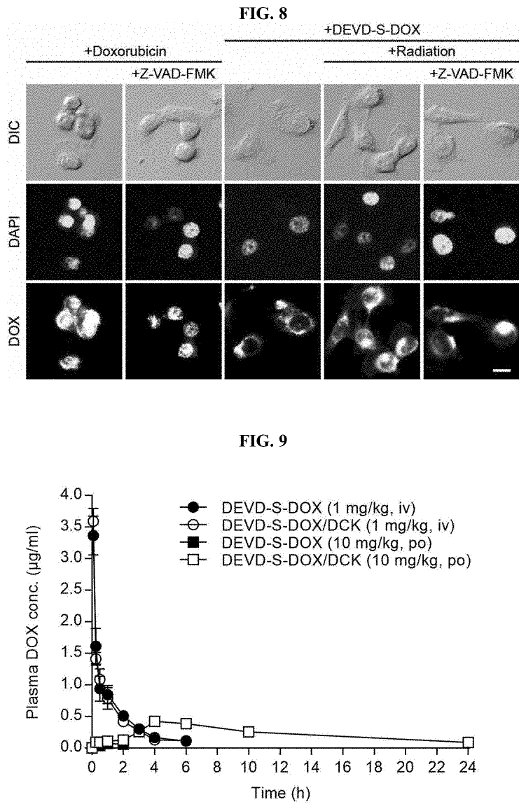

Based on the doxorubicin content, 1 mg/kg and 10 mg/kg of DEVD-S-DOX and DEVD-S-DOX/DCK were each intravenously and orally administered to Sprague Dawley rats, then blood samples were taken to quantitatively analyze drug plasma concentration. Quantitative analysis was performed by measuring the fluorescence intensity in plasma, and using a standard curve to determine concentration (FIG. 9). After oral administration, DEVD-S-DOX/DCK complex showed 16.71% bioavailability while non-complexed DEVD-S-DOX showed negligible bioavailability (Table 1), suggesting that the DCK complexation had highly contributed to oral absorption. In addition, the pharmacokinetic profiles of the two compounds after intravenous administration was very similar. Without being bound by theory, the results suggest that DEVD-S-DOX/DCK is dissociated from the complex after entering the blood stream, because the complexed form would be associated with increased hydrophobicity and blockage of ionic charges that would be expected to alter the pharmacokinetics of DEVD-S-DOX.

TABLE 1
Pharmacokinetic Parameters for Intravenous and Oral Administration
of DEVD-S-DOX and DEVD-S-DOX/DCK to SD Rats

| | | DEVD-S-DOX | | DEVD-S-DOX/DCK | |
|---|---|---|---|---|---|
| | | Intravenous | Per Oral | Intravenous | Per Oral |
| Dose | (mg/kg) | 1 | 10 | 1 | 10 |
| Tₘₐₓ | (hr) | NA | 1 | NA | 4.67 ± 0.67 |
| Cₘₐₓ | (μg/ml) | 3.37 ± 0.31 | 0.072 ± 0.033 | 3.59 ± 0.35 | 0.46 ± 0.03 |
| AUCₗₐₛₜ | (μg*hr/ml) | 3.11 ± 0.29 | 0.10 ± 0.05 | 2.98 ± 0.68 | 5.02 ± 0.21 |
| AUC_{inf} | (μg*hr/ml) | 3.37 ± 0.25 | 0.13 ± 0.06 | 3.22 ± 0.98 | 5.63 ± 0.45 |
| MRT | (h) | 2.01 ± 0.28 | 1.18 ± 0.03 | 1.70 ± 0.58 | 11.27 ± 1.55 |
| BA | (%) | — | 0.45 ± 0.22 | — | 16.71 ± 2.30 |

Tₘₐₓ, time of maximum concentration; Cₘₐₓ, maximum concentration; AUC, area under the curve; MRT, mean residence time; BA, bioavailability.
Data are presented as mean ± s.d.

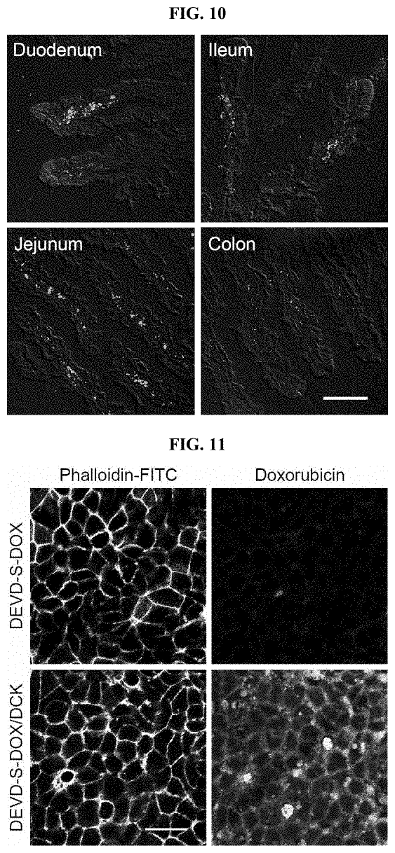

Additionally, after orally administering DEVD-S-DOX/DCK, parts of the gastrointestinal tract were extracted and observed with a florescent microscope (FIG. 10). The results showed that the drug was barely absorbed in the large intestine but evenly absorbed throughout the small intestine. Without being bound by theory, since the active transportation of bile acids mediated by bile acid transporters is restricted to the ileum, the enhanced intestinal absorption of the DEVD-S-DOX/DCK complex is considered to be mainly due to the increased overall hydrophobicity rather than interaction with intestinal bile acid transporters.

### Example 6—DEVD-S-DOX/DCK Cell Permeability in Caco-2 Cell Evaluation

Caco-2 cells were cultured on a cover-glass until completely full, then processed with DEVD-S-DOX and DEVD-S-DOX/DCK at a concentration of 5 μM for an hour. Then the cells were washed with PBS thrice, fixed with 4% PFA, and the gap junctions between cells were stained with fluorescently labeled phalloidin. The cells were then washed again with PBS thrice and then the nucleus was dyed with DAPI. By observing with a confocal fluorescence microscope, it was observed that the cell permeability of DEVD-S-DOX/DCK was much higher than that of DEVD-S-DOX (FIG. 11). When treated to the monolayer of Caco-2-cells, significantly higher absorption of DEVD-S-DOX/DCK complex was observed than for the free DEVD-S-DOX, and was found mainly in the cytoplasm rather than the tight junction, suggesting that the complexation of DCK facilitated the transcellular transportation of DEVD-S-DOX through the intestinal epithelium. As such, it was identified that the drug permeates through the transcellular pathway.

### Example 7—Evaluation of the Extent of Cell Permeability Based on DEVD-S-DOX/DCK Bile Acid Transporter Manifestation

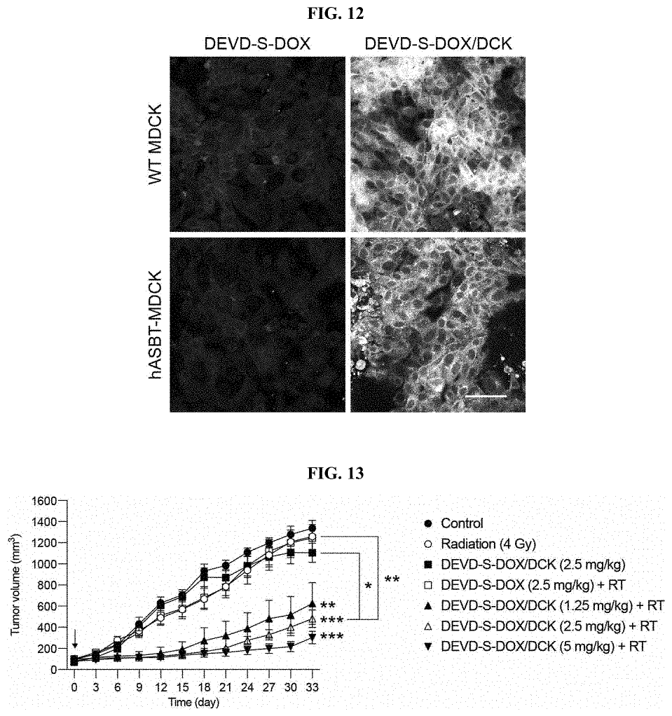

MDCK cells and ASBT-overexpressed MDCK cell (ASBT-MDCK), which is an overexpression on bile acid transporters, were cultured on a cover-glass and treated at a concentration of 5 μM for an hour. Then the cells were washed with PBS thrice and fixed with 4% PFA. By observing with a confocal fluorescence microscope (FIG. 12), it was observed that the cell permeability between the two kinds of cells differed vastly in degree and that there was no significant connection between the extent of bile acid transporter manifestation and the extent of DEVD-S-DOX/DCK permeability. The results suggest that improved intestinal epithelial cell permeability of DEVD-S-DOX/DCK was due to the increase in hydrophobicity rather than from interaction with bile acid transporter.

### Example 8—Assessment of DEVD-S-DOX/DCK Anticancer Effects in Pre-Clinical Model

Human breast cancer MDA-MB-231 cells were transplanted in a female athymic BALB/c nude mouse, and when the cancer tumor size reached 50-100 mm³, 1.25 or 5 mg/kg of DEVD-S-DOX/DCK (based on the doxorubicin content), was orally administered daily (FIG. 13). On the first day of administration, the mouse's tumor was irradiated with a 4 Gy dose of radiation. The control groups consisted of a group that was administered only saline and another that was irradiated (only) on the first day of the drug treatment. In addition, a test group without radiation was daily orally administered 2.5 mg/kg DEVD-S-DOX/DCK (dose based on the doxorubicin content).

The anticancer effect of the orally administered DEVD-S-DOX/DCK complex with or without single-dose radiotherapy was evaluated (FIG. 13). The single-dose radiotherapy (4 Gy) was ineffective in tumor growth suppression. However, when combined with daily oral administration of the DEVD-S-DOX/DCK complex, tumor growth was significantly suppressed in a dose-dependent manner, with mean tumor volume decreased by 53.2, 64.0, and 77.4% compared with the control group when administered at doses of 1.25, 2.5, and 5 mg/kg, respectively. By contrast, without radiotherapy, DEVD-S-DOX/DCK complex administration at a dose of 2.5 mg/kg showed little effect. Thus, there was a clear synergistic effect between low-dose radiotherapy and metronomic dosing of the DEVD-S-DOX/DCK complex, attributed to the cleavage/activation of the DEVD-S-DOX prodrug in the irradiated tumor. Moreover, the oral administration of free DEVD-S-DOX with radiotherapy was ineffective, indicating that DEVD-S-DOX was not absorbed orally without DCK.

Table 2 shows the hematological and biochemical parameters of the BALB/c nude mice after one month of DEVD-S-DOX/DCK administration.

TABLE 2

| | | | DEVD-S- | DEVD-S- | DEVD-S- | DEVD-S- |
|---|---|---|---|---|---|---|
| | Normal | | DOX 2.5 | DOX/DCK | DOX/DCK | DOX/DCK |
| | Range | Control | mg/kg | 1.25 mg/kg | 2.5 mg/kg | 5 mg/kg |
| WBC (m/mm³⁾ | 4.0-15.0 | 6.3 ± 1.6 | 12.8 ± 5.4 | 9.5 ± 2.4 | 14.7 ± 8.8 | 8.4 ± 1.3 |
| RBC (M/mm³) | 6.0-11.0 | 9.0 ± 0.7 | 7.1 ± 1.4 | 8.4 ± 0.5 | 6.3 ± 0.4 | 9.0 ± 0.6 |
| Hb (g/dl) | 14.0-18.0 | 13.1 ± 0.4 | 11.4 ± 1.3 | 12.8 ± 0.3 | 10.6 ± 1.0 | 13.9 ± 0.7 |
| Hct (%) | 35.0-45.0 | 45.1 ± 2.8 | 35.1 ± 7.2 | 42.6 ± 4.1 | 31.2 ± 2.6 | 47.2 ± 3.9 |
| MCV (fL) | 35.0-50.0 | 50.2 ± 2.8 | 49.2 ± 7.2 | 50.7 ± 4.1 | 49.9 ± 2.6 | 52.3 ± 3.9 |
| MCH (pg) | 13.0-22.0 | 15.2 ± 0.1 | 16.4 ± 1.8 | 15.4 ± 0.6 | 16.9 ± 1.8 | 15.4 ± 0.3 |
| MCHC (g/dL) | 24.0-40.0 | 29.1 ± 1.0 | 33.4 ± 3.8 | 30.4 ± 2.3 | 34.0 ± 3.8 | 29.5 ± 1.0 |
| Platelet (m/mm³⁾ | 600-1000 | 853 ± 298 | 1008 ± 392 | 801 ± 76 | 750 ± 220 | 616 ± 165 |
| Segment (%) | 10.0-30.0 | 7.0 ± 1.1 | 18.2 ± 0.7 | 9.5 ± 3.8 | 11.8 ± 3.4 | 7.8 ± 1.4 |
| Lympho (%) | 70.0-81.0 | 83.5 ± 3.3 | 71.0 ± 4.9 | 81.7 ± 4.4 | 73.4 ± 11.8 | 81.7 ± 2.6 |
| MONO (%) | 1.0-5.0 | 5.4 ± 1.0 | 6.8 ± 1.7 | 5.1 ± 0.4 | 5.1 ± 0.2 | 5.4 ± 0.2 |
| EOS (%) | <10.0 | 2.9 ± 0.3 | 2.8 ± 1.4 | 2.5 ± 0.6 | 9.8 ± 4.1 | 4 ± 3.6 |
| BASO (%) | % | 1.2 ± 0.6 | 1.0 ± 0.5 | 1.3 ± 0.8 | 0.6 ± 0.4 | 0.7 ± 0.2 |

Abbreviations: BASO, basophil; BUN, blood urea nitrogen; CRE, creatinine; TBIL, total bilirubin; AST, aspartate aminotransferase; ALT, alanine trans-aminase; ALK, anaplastic lymphoma kinase; CAL, calcium; WBC, white blood cell count; RBC, red blood cell count; HGB, hemoglobin; HCT, hematocrit; MCV, mean corpuscular volume; MCH, mean corpuscular hemoglobin; MCHC, mean corpuscular hemoglobin concentration; PLT, platelet; LYMPH, lymphocyte; MONO, monocyte; EOS, eosinophil.
Data are presented as mean ± s.d.

The small intestine and liver were evaluated histologically and hematologically, and no unusual pathologic feature was found. Overall, the combined use of DEVD-SDOX/DCK and radiotherapy displayed remarkable anticancer effects without serious side effects, as demonstrated in pre-clinical models.

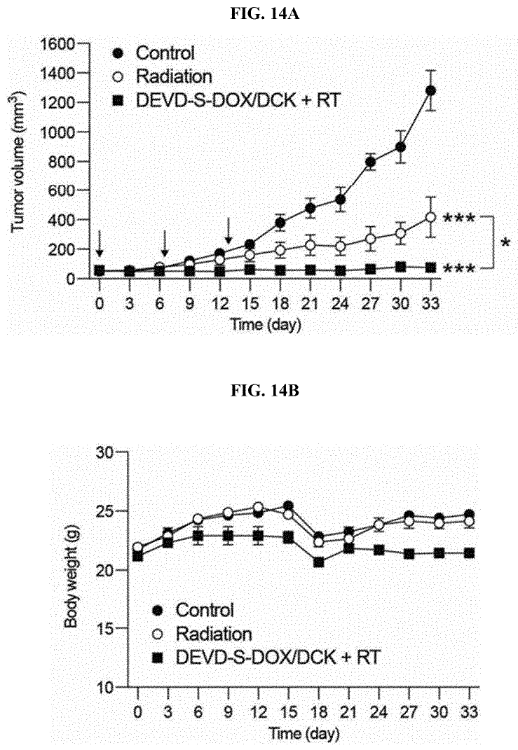

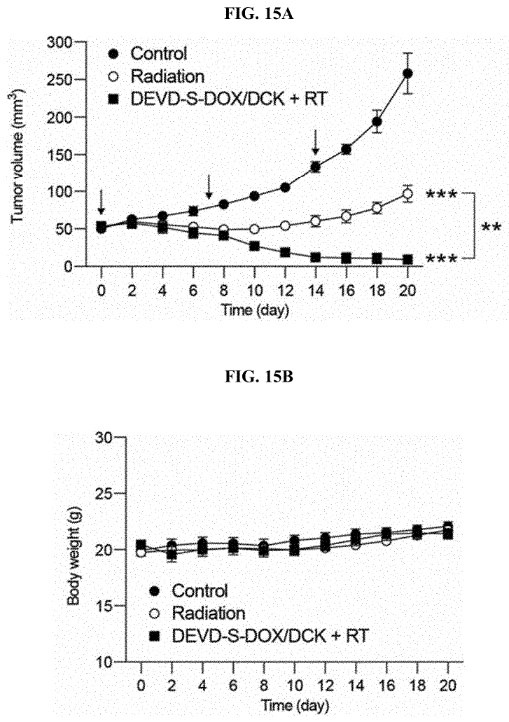

In a preclinical breast cancer model using MDA-MB-231 (FIGS. 14A and 14B) or HCC-70 (FIGS. 15A and 15B) xenografts, a 5 mg/kg dose of DEVD-S-DOX/DCK (based on doxorubicin content) was used with weekly irradiation (3 cycles once a week), rather than a single irradiation (FIGS. 14A and 14B). The weekly dosing of radiotherapy was intended to maintain a sufficient level of intratumoral caspase-3 to activate the DEVD-S-DOX over the long-term.

Unlike single-dose radiotherapy, multiple-dose radiotherapy showed potent anticancer effects by itself, decreasing tumor volume by 67.4 and 62.5% compared with control in MDA-MB-231 and HCC-70 xenografts, respectively. Still, combining multiple-dose radiotherapy with oral DEVD-S-DOX/DCK complex had an even greater effect, and highly suppressed tumor growth, decreasing tumor volume by 94.1% and 96.4% compared to control in MDA-MB-231 and HCC-70 xenografts, respectively. Moreover, despite the potent anticancer effect, no apparent systemic toxicities were observed, as reflected in the body-weight profiles shown in FIG. 14B and FIG. 15B and blood test results (Table 2). Although some of the hematological and biochemical parameters in mice that received radiotherapy and DEVD-S-DOX/DCK were slightly out of the normal range, their values were similar to that of the control group.

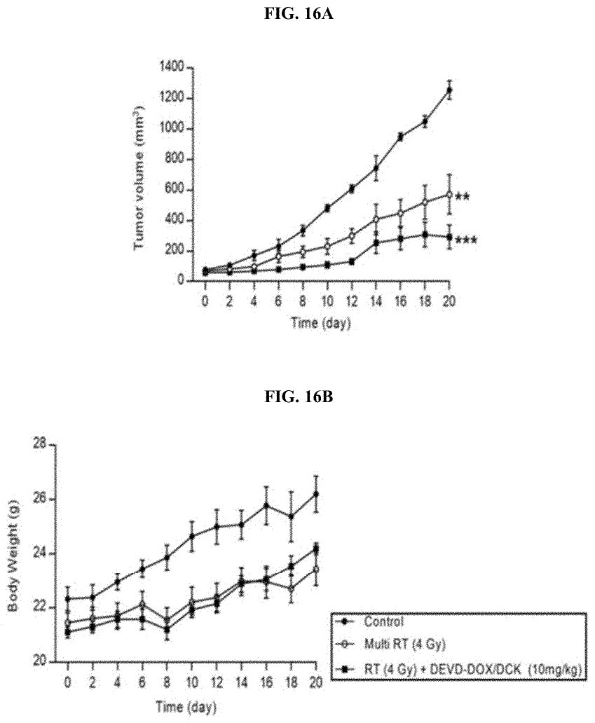

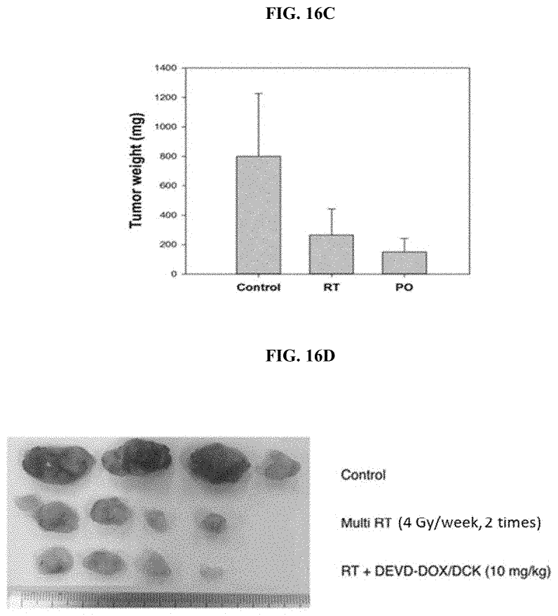

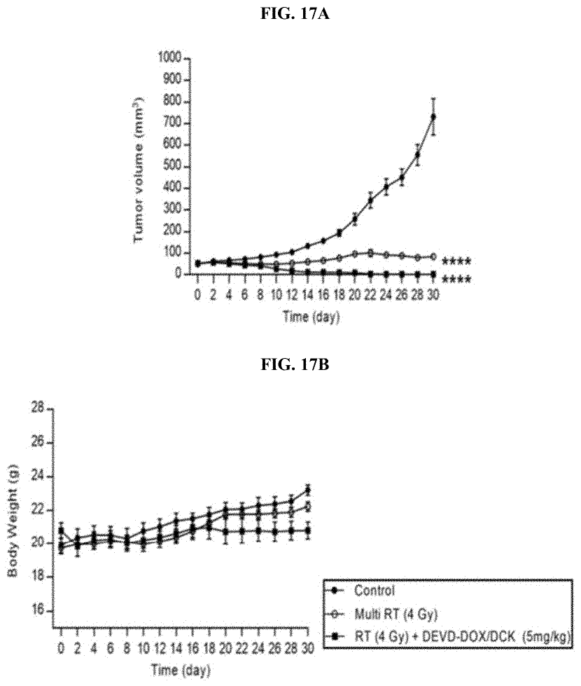

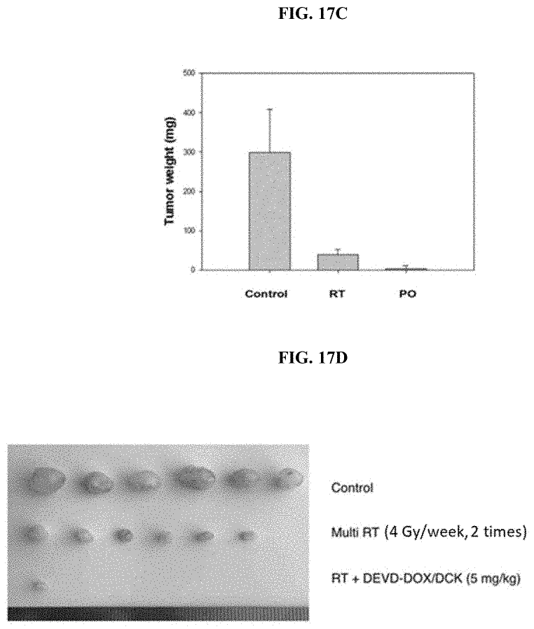

The experiment above was repeated with a different pre-clinical breast cancer model (HCC-70) (FIGS. 17A-17D) over a period of 30 days (versus a period of 20 days depicted in FIGS. 15A and 15B) and pancreatic cancer pre-clinical model (Pan02) (FIGS. 16A-16D). For these studies, a 5 mg/kg dose of DEVD-S-DOX/DCK for HCC-70 (based on doxorubicin content) or a 10 mg/kg dose of DEVD-S-DOX/DCK for Pan02 (based on doxorubicin content) was coupled with repeated-dose radiation, The drugs were administered daily while radiation was given at a dose of 4 Gy at day 0 and day 7. The results showed that the group treated with orally administered DEVD-S-DOX/DCK and radiation therapy displayed more significant anticancer effects when compared to both the control and the test groups administered radiotherapy alone. Also, the size of the cancer tumors was reduced with continued DEVD-S-DOX/DCK administration.

Our results show that oral DEVD-S-DOX/DCK complex treatment coupled with single-dose radiotherapy resulted in tumor regrowth after 2-3 weeks of therapy. We hypothesized the regrowth was due to the failure to maintain a sufficient amount of intratumoral caspase-3 to cleave DEVD-S-DOX in the tumor. Accordingly, radiotherapy at repeated doses was administered in order to continuously induce the upregulation of caspase-3. Consequently, tumor growth was effectively controlled over a month, showing no evident growth during the observation. This suggested that the repeated low-dose radiotherapy resulted in maintaining a sufficient amount of caspase-3 in the tumor and allowed continuous activation/cleavage of the DEVD-S-DOX/DCK complex or DEVD-S-DOX conjugate over long term treatment. The daily administration of the DEVD-S-DOX/DCK complex at 5 mg/kg (based on the DOX content) for over a month, which was >150 mg/kg of accumulated dose of doxorubicin, did not cause any significant systemic toxicity, according to the body weight profile and blood test results. In addition, the histopathological analysis showed no sign of significant toxicity in the gastrointestinal tract and liver, which are in the absorption route of the prodrug before entering the systemic circulation. Considering that the orally administered prodrug was ineffective without radiotherapy, our results indicate that DEVD-S-DOX successfully maintained its property as a prodrug during oral absorption, which is important for it to function as in radiation-induced apoptosis-targeted chemotherapy (RIATC).

### Example 9—Synthesis of DEVD-S-DCX/DCK

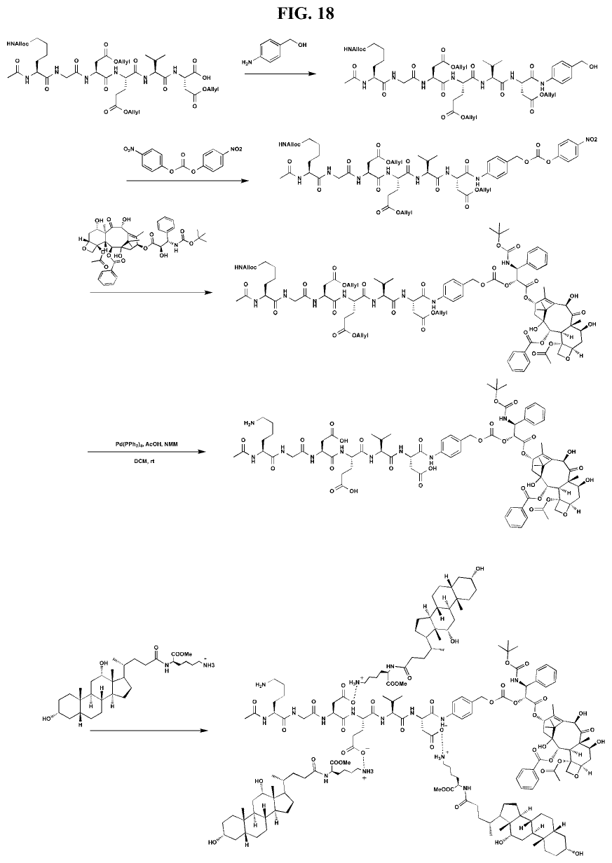

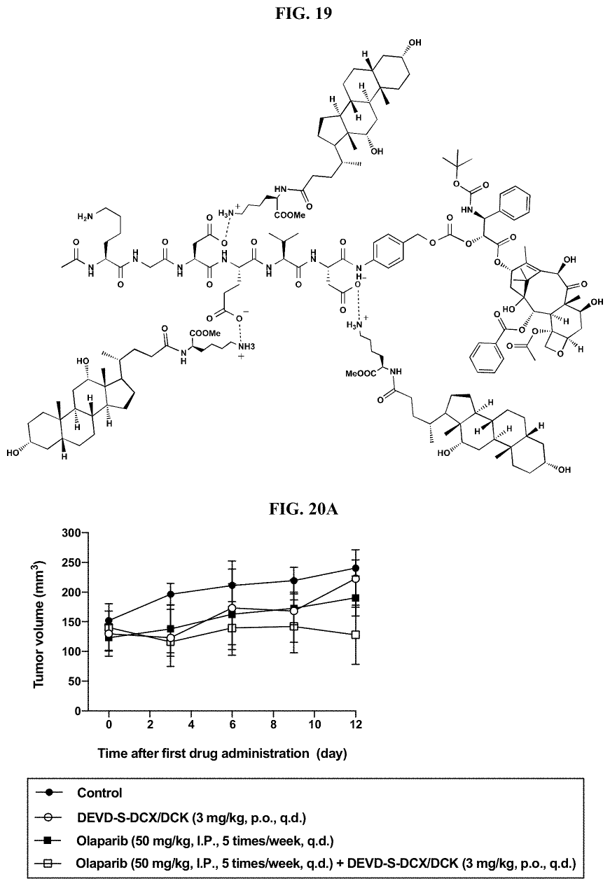

FIG. 18 shows a synthesis scheme for the preparation described below of the DEVD-S-DCX/DCK complex and FIG. 19 shows the structure of the DEVD-S-DCX/DCK complex. AcK(OAlloc)GD(OAll)E(OAll)VD(OAll)-OH (600 mg, 0.55 mmol, 1 equivalent), p-aminobenzyl alcohol (137 mg, 1.1 mmol, 2 molar equivalents), and EEDQ (275 mg, 1.1 mmol, 2 molar equivalents) were dissolved in anhydrous DMF (Sigma Aldrich, STBH6050/30 mL) and filled N₂ gas in flask. The reaction mixture was stirred for 16 hours at room temperature. After the completion of reaction was confirmed by HPLC, the solution was concentrated (5 mL) and poured in to cool diethyl ether (100 mL) to form precipitate. The precipitate was collected by filtration and washed cool with diethyl ether (20 mL×6 times). The collected precipitate was dried in vacuo to obtain AcK(OAlloc)GD(OAll)E(OAll)VD(OAll)-PABA as a white powder (551 mg, 76.5%).

AcK(OAlloc)GD(OAll)E(OAll)VD(OAll)-PABA (551 mg, 0.54 mmol, 1 molar equivalent), bis(p-nitrophenyl)carbonate (827 mg, 2.7 mmol, 5 molar equivalents) were dissolved in anhydrous DMF (20 mL) and filled N₂ gas in flask. DIPEA (285 μL, 1.62 mmol, 3 equivalents) was added dropwise to the solution. The reaction mixture was stirred for 18 hours at room temperature. After the completion of reaction was confirmed by HPLC, the solution was concentrated (5 mL) and poured in to cool diethyl ether (100 mL) to form precipitate. The precipitate was collected by filtration and washed with diethyl ether (20 mL×6 times). The collected precipitate was dried in vacuo to obtain AcK(OAlloc)GD(OAll)E(OAll)VD(OAll)-PABC as a yellow solid (490 mg, 91.9%).

AcK(OAlloc)GD(OAll)E(OAll)VD(OAll)-PABC (490 mg, 0.42 mmol, 1 equivalent) and docetaxel (403 mg, 0.5 mmol, 1.2 molar equivalents) were dissolved in anhydrous DMF (15 mL) under N₂ atmosphere. DMAP (152 mg, 1.25 mmol, 3 equivalents) was added slowly and stirred for 16 hours at room temperature. After the completion of reaction was confirmed by HPLC, the reaction mixture was concentrated (5 mL) and precipitated in cold diethyl ether (100 ml) during stirring. The precipitate was filtered using a membrane filter and washed with cold diethyl ether (30 mL×8 times) and cold ethanol (20 mL×3 times) in sequence. The collected precipitate was dried in vacuo to obtain AcK(OAlloc)GD(OAll)E(OAll)VD(OAll)-PABC-DCX as a yellow solid (767 mg, 75.1%).

AcK(OAlloc)GD(OAll)E(OAll)VD(OAll)-PABC-DCX (767 mg, 0.42 mmol, 1 equivalent) was dissolved in dichloromethane (111 mL)/AcOH (6 mL)/NMM (3 mL) (v/v/v=37:2:1). The flask was filled with N₂ gas and degassed before adding the catalyst. Pd(PPh₃)₄ (1440 mg, 1.29 mmol, 3 equivalents) was dissolved in dichloromethane (2 mL) and added slowly. The reaction mixture was stirred for 4 hours at room temperature. Deprotected AcKGDEVD-PABC-DCX precipitate appeared as the reaction was proceeded. To confirm that the reaction was finished, the precipitated was filtered to using membrane filter and washed with cold dichloromethane (30 mL×4 times). The precipitate was dried in vacuo to obtain AcKGDEVD-PABC-DCX as a powder (415 mg, 75.6%).

The precipitate (415 mg) was dissolved in ACN 5% (in DDW, 0.1% TFA as an additive) and subjected to semi-preparative reverse-phase HPLC (Shimadzu, Kyoto, Japan) using an octadecylsilyl (ODS-A) 5 μm semi-preparative column for further purification (125 mg, 98.0%). ESI-MS: m/z 1642.69 [M+H]+.

DEVD-S-DCX (50 mg, 0.03 mmol) and DCK (50 mg, 0.09 mmol, 3 equivalents) were dissolved in distilled water and 0.1N NaHCO₃ was added to adjust solution's pH to 7. Labrasol and poloxamer 188 was introduced to the solution as surfactant.

### Example 10—Assessment of DEVD-S-DCX/DCK Anticancer Effects in Pre-Clinical Model

Human breast cancer MDA-MB-436 cells were transplanted into a female NSG SCID mouse, and when the cancer tumor size reached 100-150 mm³, 3 mg/kg of DEVD-S-DCX/DCK (based on the docetaxel content), was orally administered daily. 50 mg/kg of olaparib was administered by intraperitoneal injection daily for the first five days of every week for two weeks (5 days on/2 days off). In addition, a test group without olaparib was daily orally administered 3 mg/kg DEVD-S-DCX/DCK (based on the docetaxel content).

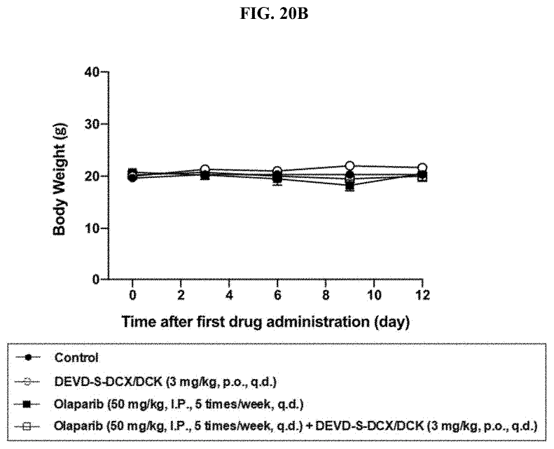

The anticancer effect of the orally administered DEVD-S-DCX/DCK complex with or without olaparib treatment was evaluated. FIGS. 20A and 20B depict the tumor volume profiles and body weight profiles, respectively. DEVD-S-DCX/DCK or olaparib monotherapy was ineffective in tumor growth suppression. However, when DEVD-S-DCX/DCK was combined with daily administration of olaparib, it produced tumor growth inhibition (TGI) of 114.9% (P<0.01). Thus, there was a clear synergistic effect between olaparib and metronomic dosing of the DEVD-S-DCX/DCK complex. This result indicates that tumor targeted therapy by olaparib was effective for inducing cancer cell apoptosis, which enabled the DEVD-S-DCX/DCK complex to be activated by expressed caspase-3 at the tumor site.

1. A complex comprising:
(a) an anticancer prodrug moiety comprising (i) a caspase-cleavable peptide, covalently attached directly or through a linker, to (ii) an anticancer chemotherapeutic agent; and
(b) a bile acid moiety, wherein the bile acid moiety is non-covalently complexed to the anticancer prodrug moiety.

2. The complex of claim 1, wherein the caspase-cleavable peptide is cleavable by a caspase selected from caspase-3, caspase-7, and caspase-9.

3. The complex of claim 1, wherein the caspase-cleavable peptide comprises an amino acid sequence selected from Asp-Glu-Val-Asp (SEQ ID NO:4), Asp-Leu-Val-Asp (SEQ ID NO:5), Asp-Glu-Ile-Asp (SEQ ID NO:6), and Leu-Glu-His-Asp (SEQ ID NO:7).

4. The complex of claim 1, wherein the caspase-cleavable peptide comprises the amino acid sequence Asp-Glu-Val-Asp (SEQ ID NO:4).

5. The complex of claim 1, wherein the caspase-cleavable peptide is covalently attached to the anticancer chemotherapeutic agent through a linker selected from para-aminobenzyloxycarbonyl, aminoethyl-N-methylcarbonyl, aminobiphenylmethyloxycarbonyl, a dendritic linker and a cephalosporin-based linker.

6. The complex of claim 1, wherein the caspase-cleavable peptide is covalently attached to the anticancer chemotherapeutic agent through a para-aminobenzyloxycarbonyl linker.

7. The complex of claim 1, wherein the anticancer chemotherapeutic agent is selected from anthracyclines, antibiotics, alkylating agents, platinum-based agents, antimetabolites, topoisomerase inhibitors, and mitotic inhibitors.

8. The complex of claim 1, wherein the anticancer chemotherapeutic agent is selected from doxorubicin, daunorubicin, epirubicin, idarubicin, valrubicin, anthracyclin; actinomycin-D, bleomycin, mitomycin-C, cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine; camptothecin, topotecan, irinotecan, etoposide, teniposide, mitoxantrone, paclitaxel, docetaxel, izabepilone, vinblastine, vincristine, vindesine, vinorelbine, estramustine, and derivatives thereof.

9. The complex of claim 1, wherein the anticancer chemotherapeutic agent is selected from doxorubicin, daunorubicin, and docetaxel.

10. The complex of claim 1, wherein the bile acid moiety is selected from cholic acid, deoxycholic acid, chenodeoxycholic acid, lithocholic acid, ursocholic acid, ursodeoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, and hyodeoxycholic acid.

11. The complex of claim 1, wherein the bile acid moiety is Na-deoxycholyl-L-lysine-methylester.

12. The complex of claim 1, wherein a plurality of bile acid moieties is non-covalently complexed to a single anticancer prodrug moiety.

13. The complex of claim 1, wherein the bile acid moiety is non-covalently complexed to the anticancer prodrug moiety by ionic bonding, hydrophobic bonding, or coordinate bonding.

14. The complex of claim 1, wherein:
(a) the anticancer prodrug moiety comprises (i) a caspase-cleavable peptide comprising the amino acid sequence Asp-Glu-Val-Asp (SEQ ID NO:4) joined through a para-aminobenzyloxycarbonyl linker to (ii) an anticancer chemotherapeutic agent; and
(b) the bile acid moiety comprises N^{α}-deoxycholyl-L-lysine-methylester.

15. An oral pharmaceutical composition comprising the complex of claim 1 and a pharmaceutically acceptable carrier for oral administration.

16. A method of inducing amplified apoptosis of tumor cells in a subject, comprising:
(a) administering to a subject in need thereof an apoptosis-inducing treatment effective to induce expression of caspase in tumor cells, and
(b) orally administering to the subject a complex according to claim 1.

17. The method of claim 16, wherein the apoptosis-inducing treatment is selected from radiotherapy, hyperthermia, laser therapy, photodynamic therapy, chemotherapy, and cryosurgery.

18. The method of claim 16, comprising weekly administration of radiotherapy at a dose of 1 to 35 Gy, and daily administration of the complex at a dose of 1 to 100 mg/kg, based on the molar equivalent dose of the anticancer chemotherapeutic agent.

19. The method of claim 16, wherein the method results in gastrointestinal absorption of the chemotherapeutic prodrug of the complex.

20. The method of claim 16, wherein the apoptosis-inducing treatment comprises chemotherapy, optionally comprising administration of olaparib, trastzumab, ado-trastuzumab emtansine, tamoxifen, lapatinib, palbociclib, ribociclib, neratinib maleate, or abemaciclib.


SEQUENCE LISTING (9 sequences)

SEQ ID NO: 1 - protein, 4 aa, Artificial Sequence
  Description of Artificial Sequence Synthetic peptide
  MOD_RES (2)..(3): Any amino acid, in L- or D- isomer form
DXXD

SEQ ID NO: 2 - protein, 4 aa, Artificial Sequence
  Description of Artificial Sequence Synthetic peptide
  MOD_RES (2)..(3): Any amino acid, in L- or D- isomer form
LXXD

SEQ ID NO: 3 - protein, 4 aa, Artificial Sequence
  Description of Artificial Sequence Synthetic peptide
  MOD_RES (2)..(3): Any amino acid, in L- or D- isomer form
VXXD

SEQ ID NO: 4 - protein, 4 aa, Artificial Sequence
  Description of Artificial Sequence Synthetic peptide
DEVD

SEQ ID NO: 5 - protein, 4 aa, Artificial Sequence
  Description of Artificial Sequence Synthetic peptide
DLVD

SEQ ID NO: 6 - protein, 4 aa, Artificial Sequence
  Description of Artificial Sequence Synthetic peptide
DEID

SEQ ID NO: 7 - protein, 4 aa, Artificial Sequence
  Description of Artificial Sequence Synthetic peptide
LEHD

SEQ ID NO: 8 - protein, 6 aa, Artificial Sequence
  Description of Artificial Sequence Synthetic peptide
KGDEVD

SEQ ID NO: 9 - protein, 6 aa, Artificial Sequence
  Description of Artificial Sequence Synthetic peptide
  MOD_RES (1)..(1): Lys(OAlloc)
  MOD_RES (3)..(3): Asp(OAll)
  MOD_RES (4)..(4): Glu(OAll)
  MOD_RES (6)..(6): Asp(OAll)
KGDEVD